(19) United States
(12) Patent Application Publication
(10) Pub. No.: US 2022/0168036 A1
(43) Pub. Date: Jun. 2, 2022

(54) SYSTEMS, APPARATUSES, AND METHODS FOR REMOVING A MEDICAL IMPLANT FROM CARDIAC TISSUE

(71) Applicant: Evalve, Inc., Santa Clara, CA (US)

(72) Inventor: Michael F. Wei, Redwood City, CA (US)

(21) Appl. No.: 17/532,590

(22) Filed: Nov. 22, 2021

Related U.S. Application Data
(60) Provisional application No. 63/119,352, filed on Nov. 30, 2020.

(51) Int. Cl. A61B 18/14

(52) U.S. Cl. CPC A61B 18/14; A61B 2018/00083

(57) ABSTRACT

An implant removal device having an elongate body having a proximal end and a distal end, the elongate body being resiliently flexible and configured to transmit torque from the proximal end to the distal end with a predetermined turning ratio, and a capture structure extending distally from the distal end and having a capture region, the capture structure being configured to selectively center a deployed implant in relation to a longitudinal axis of the elongate body and the capture region to aid with capture and subsequent removal.

### CROSS-REFERENCE TO RELATED APPLICATIONS

This application claims benefit and priority to U.S. Provisional Patent Application No. 63/119,352, filed Nov. 30, 2020, entitled “SYSTEMS, APPARATUSES, AND METHODS FOR REMOVING A MEDICAL IMPLANT FROM CARDIAC TISSUE,” the entire contents of which are incorporated by reference herein.

### BACKGROUND OF THE INVENTION

### 1. The Field of the Invention

The present disclosure relates generally systems, apparatuses, and methods for removal of a medical implant from cardiac tissue, such as a medical implant attached to a valve leaflet.

### 2. The Relevant Technology

The present invention relates generally to medical methods, devices, and systems. In particular, the present invention relates to methods, devices, and systems for the endovascular, percutaneous, or minimally invasive surgical treatment of bodily tissues, such as tissue approximation or valve repair. More particularly, the present invention relates to repair of valves of the heart and venous valves, and devices and methods for removing or disabling mitral valve repair components through minimally invasive procedures.

Surgical repair of bodily tissues often involves tissue approximation and fastening of such tissues in the approximated arrangement. When repairing valves, tissue approximation includes coapting the leaflets of the valves in a therapeutic arrangement which may then be maintained by fastening or fixing the leaflets. Such coaptation can be used to treat regurgitation which most commonly occurs in the mitral valve.

Mitral valve regurgitation is characterized by retrograde flow from the left ventricle of a heart through an incompetent mitral valve into the left atrium. During a normal cycle of heart contraction (systole), the mitral valve acts as a check valve to prevent flow of oxygenated blood back into the left atrium. In this way, the oxygenated blood is pumped into the aorta through the aortic valve. Regurgitation of the valve can significantly decrease the pumping efficiency of the heart, placing the patient at risk of severe, progressive heart failure.

Mitral valve regurgitation can result from a number of different mechanical defects in the mitral valve or the left ventricular wall. The valve leaflets, the valve chordae which connect the leaflets to the papillary muscles, the papillary muscles themselves or the left ventricular wall may be damaged or otherwise dysfunctional. Commonly, the valve annulus may be damaged, dilated, or weakened, limiting the ability of the mitral valve to close adequately against the high pressures of the left ventricle.

The most common treatments for mitral valve regurgitation rely on valve replacement or repair including leaflet and annulus remodeling, the latter generally referred to as valve annuloplasty. One technique for mitral valve repair which relies on suturing adjacent segments of the opposed valve leaflets together is referred to as the “bow-tie” or “edge-to-edge” technique. While all these techniques can be effective, they usually rely on open heart surgery where the patient's chest is opened, typically via a sternotomy, and the patient placed on cardiopulmonary bypass. The need to both open the chest and place the patient on bypass is traumatic and has associated high mortality and morbidity.

In some patients, a fixation device can be installed into the heart using minimally invasive techniques. The fixation device can hold the adjacent segments of the opposed valve leaflets together and may reduce mitral valve regurgitation. One such device used to clip the anterior and posterior leaflets of the mitral valve together is the MitraClip® fixation device, sold by Abbott Vascular, Santa Clara, Calif., USA.

However, sometimes after a fixation device is installed, undesirable mitral valve regurgitation can still exist, or can arise again. Further, other problems with the heart may arise that can make it desirable for the fixation device to be disabled or removed, usually in order that other procedures may be performed on the heart.

Current techniques for removing or disabling mitral valve fixation devices usually rely on open heart surgery where the patient's chest is opened, typically via a sternotomy, and the patient placed on cardiopulmonary bypass.

For these reasons, it would be desirable to provide alternative and additional methods, devices, and systems for removing or disabling fixation devices that are already installed. Such methods, devices, and systems should preferably not require open chest access and be capable of being performed either endovascularly, i.e., using devices which are advanced to the heart from a point in the patient's vasculature remote from the heart or by another minimally invasive approach. The methods, devices, and systems may be useful for repair of tissues in the body other than heart valves. At least some of these objectives will be met by the inventions described hereinbelow.

### BRIEF SUMMARY OF THE INVENTION

These and other objects and features of the present invention will become more fully apparent from the following description and appended claims or may be learned by the practice of the invention as set forth hereinafter.

The present disclosure describes methods and devices that may be employed after a device that clips the anterior and posterior leaflets of the mitral valve together has been installed.

Sometimes after such a device is installed in the heart, problems may still exist or could arise with the function of the mitral valve or with the heart generally. In order to resolve these problems, it may be desirable to remove or disable the previously implanted device. It may also be desirable to perform a procedure on the mitral valve, such as mitral valve annuloplasty, balloon valvuloplasty, mitral valve repair, or installation of a replacement valve. In order to be able to perform procedures on a heart that already has a mitral valve fixation device attached thereto, it may be desirable to first remove or disable the device.

Traditionally, mitral valve fixation devices have been removed through invasive surgeries, such as open-heart surgery. However, less invasive methods would be preferable, because, for example, persons with a mitral valve fixation device may not be suitable candidates for an invasive surgery. Disclosed herein are methods and devices that may be used in disabling or removing such a device.

For example, according to an embodiment, a method of removing a fixation device that holds anterior and posterior leaflets of the mitral valve together is disclosed. The method may include surrounding a portion of the fixation device with a capture structure, enclosing the captured fixation device, separating the fixation device from the cardiac tissue, and removing the fixation device from the patient.

According to another embodiment, a method of removing a fixation device may include cutting one leaflet along or near the engagement of the fixation device with the leaflet so that the fixation device separates from a main portion of that leaflet from which it is cut.

Another method for removing a fixation device may include accessing, through an endovascular procedure, the fixation device holding the anterior and posterior leaflets of the mitral valve together. The endovascular procedure may advance a capture structure, such as a portion of a coiled structure, through the vasculature of the patient, and into the heart. Following capturing a portion of the fixation device with the capture structure, the fixation device may be separated (e.g., cut) from both leaflets with a removal tool that at least partially surrounds the capture structure and the fixation device. The fixation device may then be removed from the body of the patient.

Any of such described methods may advantageously be performed with minimal invasion, e.g., through an endovascular procedure that advances any devices employed in the procedure (e.g., tools for capturing the fixation device, cutting or otherwise separating the fixation device and/or surrounding tissue) through the vasculature of the patient, into the heart, where the devices may access the mitral valve.

Another embodiment according to the present disclosure is directed to a system for removing a mitral valve fixation device. The system may include an implant management tool with cutting means disposed at the distal end, the cutting means being configured to cut the tissue surrounding the installed fixation device. The system may further include an implant removal device with a capture structure, such as a retaining means, disposed at the distal end. The capture structure may be configured to retain the fixation device and/or cut portions thereof, so as to allow its removal using the implant management tool.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates the left ventricle and left atrium of the heart during systole.

FIG. 2A illustrates free edges of leaflets of the mitral valve in normal coaptation, and FIG. 2B illustrates the free edges in regurgitative coaptation.

FIG. 3 illustrates the position of the fixation device in a desired orientation relative to the leaflets.

FIG. 4 is another illustration of the position of the fixation device in a desired orientation relative to the leaflets.

FIG. 5 is an illustration of the position of the fixation device in a desired orientation relative to the leaflets following tissue ingrowth.

FIG. 6A-B illustrate a system for removing a fixation device according to one configuration of the invention.

FIG. 7A-7I illustrate various configurations of a cutting member according to various configurations of the invention.

FIG. 8 is a cross-sectional view of an implant removal device of the system of FIG. 6A-B according to one configuration of the invention.

FIG. 9 is a partial cross-section view of an implant removal device of the system of FIG. 6A-B including a sheath according to one configuration of the invention.

FIG. 10A-10F illustrate alternate cross-sections for a coiled member according to configurations of the invention.

FIG. 11 illustrates an alternate configuration of an implant removal device of according to one configuration of the invention.

FIG. 12 illustrates an alternate configuration of an implant removal device of according to one configuration of the invention.

FIG. 13 illustrates an alternate configuration of an implant removal device of according to one configuration of the invention.

FIG. 14 is a cut-away view of a heart with a portion of an implant management tool according to the present invention.

FIG. 15 a cut-away view of a heart with a portion of an implant management tool according to the present invention.

FIG. 16 a cut-away view of a heart with a portion of an implant management tool according to the present invention

FIG. 17 illustrates an implant management tool advanced toward a fixation device that was previously implanted on leaflets.

FIG. 18 illustrates an implant removal tool advanced from the implant management tool towards the fixation device of FIG. 17.

FIG. 19 illustrates a capture structure of an implant removal tool capturing the fixation device of FIG. 17.

FIG. 20 illustrates an implant removal tool, with captured fixation device, with withdrawn into the implant management tool of FIG. 17.

FIG. 21 illustrates the captured fixation device being cut from the leaflets and become disposed within a portion of the implant management tool.

FIG. 22 illustrates an alternate configuration of an implant removal device of according to one configuration of the invention.

FIG. 23 illustrates an alternate configuration of an implant removal device of according to one configuration of the invention.

FIG. 24 illustrates an implant management tool advanced toward a fixation device that was previously implanted on leaflets.

FIG. 25 illustrates an implant removal tool advanced from the implant management tool towards the fixation device of FIG. 24.

FIG. 26 illustrates a capture structure of an implant removal tool capturing the fixation device of FIG. 24.

FIG. 27 illustrates an implant removal tool, with captured fixation device, with withdrawn into the implant management tool of FIG. 24.

FIG. 28 illustrates the captured fixation device being cut from the leaflets and become disposed within a portion of the implant management tool.

### DETAILED DESCRIPTION OF THE INVENTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to apparatuses, systems, and methods to remove a fixation device deployed to a target location. The apparatuses, systems, and methods can be used to separate a fixation device coapting leaflet tissue, such as the mitral valve leaflets, and then remove the fixation device. Following fixation device removal, a replacement valve or other device can subsequently be deployed to replace the mitral valve, for instance. Illustrative fixation devices can include, but not limited to, MitraClip®.

While the present disclosure will describe a particular implementation of apparatuses and systems, with associated methods, for removing the fixation device, it should be understood that any of systems, apparatuses, and methods described herein may be applicable to other uses, including and not limited to removing fixation devices positioned in other locations with a patient's anatomy. Additionally, elements described in relation to any embodiment depicted and/or described herein may be combinable with elements described in relation to any other embodiment depicted and/or described herein.

### I. Introduction

### A. Cardiac Physiology

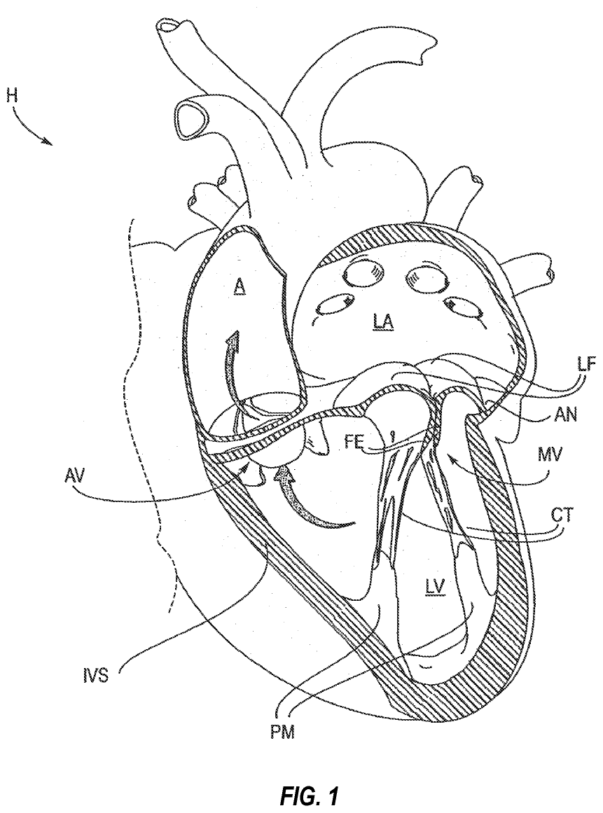

The left ventricle (LV) of a normal heart H in systole is illustrated in FIG. 1. The left ventricle (LV) is contracting and blood flows outwardly through the tricuspid (aortic) valve (AV) in the direction of the arrows. Back flow of blood or “regurgitation” through the mitral valve (MV) is prevented since the mitral valve is configured as a “check valve” which prevents back flow when pressure in the left ventricle is higher than that in the left atrium (LA). The mitral valve (MV) comprises a pair of leaflets having free edges (FE) which meet evenly to close, as illustrated in FIG. 1. The opposite ends of the leaflets (LF) are attached to the surrounding heart structure along an annular region referred to as the annulus (AN). The free edges (FE) of the leaflets (LF) are secured to the lower portions of the left ventricle LV through chordae tendinae (CT) (referred to hereinafter as the chordae) which include a plurality of branching tendons secured over the lower surfaces of each of the valve leaflets (LF). The chordae (CT) in turn, are attached to the papillary muscles (PM) which extend upwardly from the lower portions of the left ventricle and intraventricular septum IVS.

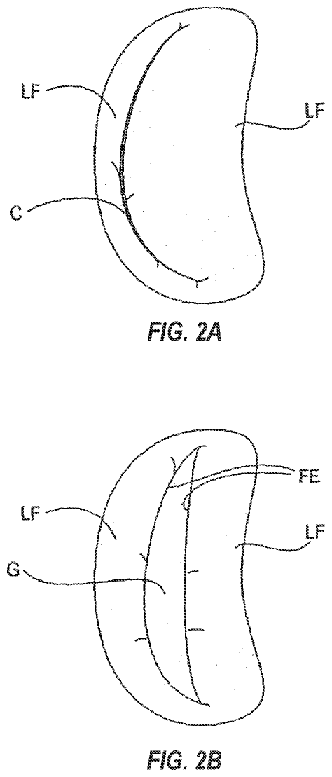

A number of structural defects in the heart can cause mitral valve regurgitation. Regurgitation occurs when the valve leaflets do not close properly allowing leakage from the ventricle into the atrium. As shown in FIG. 2A, the free edges of the anterior and posterior leaflets normally meet along a line of coaptation (C). An example of a defect causing regurgitation is shown in FIG. 2B. Here an enlargement of the heart causes the mitral annulus to become enlarged, making it impossible for the free edges (FE) to meet during systole. This results in a gap (G) which allows blood to leak through the valve during ventricular systole. Ruptured or elongated chordae can also cause a valve leaflet to prolapse since inadequate tension is transmitted to the leaflet via the chordae. While the other leaflet maintains a normal profile, the two valve leaflets do not properly meet and leakage from the left ventricle into the left atrium will occur. Such regurgitation can also occur in patients who have suffered ischemic heart disease where the left ventricle does not contract sufficiently to effect proper closure.

### II. General Overview of Fixation Technology

Fixation devices are used for grasping, approximating and fixating tissues such as valve leaflets to treat cardiac valve regurgitation, particularly mitral valve regurgitation. In some cases, the fixation devices may also provide features that allow repositioning and removal of the device if so desired, particularly in areas where removal may be hindered by anatomical features such as chordae CT. Such removal would allow the surgeon to reapproach the valve in a new manner if so desired.

Grasping will preferably be atraumatic providing a number of benefits. By atraumatic, it is meant that the devices and methods may be applied to the valve leaflets and then removed without causing any significant clinical impairment of leaflet structure or function. The leaflets and valve continue to function substantially the same as before the fixation devices are applied. Thus, some minor penetration or denting of the leaflets may occur using the devices while still meeting the definition of “atraumatic.” Similarly, during disabling or removal of the fixation device, a small portion of the leaflet(s) may be cut around the edges of the fixation device. Such atraumatic installation, disabling, or removal enables the devices to be applied to a diseased valve and, if desired, removed or repositioned without having negatively affected valve function. In addition, it will be understood that in some cases it may be necessary or desirable to pierce or otherwise permanently affect the leaflets during either grasping, fixing and/or removal. In some cases, grasping and fixation may be accomplished by a single device.

The fixation devices may rely upon the use of an interventional tool that is positioned near a desired treatment site and used to grasp the target tissue. In endovascular applications, the interventional tool is typically an interventional catheter. In surgical applications, the interventional tool is typically an interventional instrument. Fixation of the grasped tissue is accomplished by maintaining grasping with a portion of the interventional tool which is left behind as an implant. The fixation devices are well adapted for the repair of valves, especially cardiac valves such as the mitral valve.

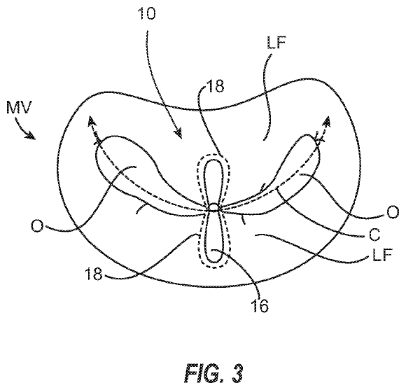

FIG. 3 illustrates the position of a fixation device 10 in a desired orientation in relation to the leaflets LF. Additional description regarding such fixation may be found in U.S. Pat. No. 9,572,666, the disclosure of which is incorporated herein by reference in its entirety. The illustrated view is a short-axis view of the mitral valve MV from the atrial side, therefore, the proximal elements 12 are shown in solid line and the distal elements 14 are shown in dashed line. When describing the devices of the invention herein, “proximal” shall mean the direction toward the end of the device to be manipulated by the user outside the patient's body, and “distal” shall mean the direction toward the working end of the device that is positioned at the treatment site and away from the user. With respect to the mitral valve, proximal shall refer to the atrial or upstream side of the valve leaflets and distal shall refer to the ventricular or downstream side of the valve leaflets.

The proximal and distal elements 12, 14 are positioned to be substantially perpendicular to the line of coaptation C. The device 10 may be moved roughly along the line of coaptation to the location of regurgitation. The leaflets LF are held in place so that during diastole, as shown in FIG. 3, the leaflets LF remain in position between the elements 12, 14 surrounded by openings or orifices O which result from the diastolic pressure gradient. Advantageously, leaflets LF are coapted such that their proximal or upstream surfaces are facing each other in a vertical orientation, parallel to the direction of blood flow through mitral valve MV. The upstream surfaces may be brought together so as to be in contact with one another or may be held slightly apart, but will preferably be maintained in the vertical orientation in which the upstream surfaces face each other at the point of coaptation. This simulates the double orifice geometry of a standard surgical bow-tie repair. Color Doppler echo will show if the regurgitation of the valve has been reduced. If the resulting mitral flow pattern is satisfactory, the leaflets may be fixed together in this orientation. If the resulting color Doppler image shows insufficient improvement in mitral regurgitation, the fixation device 10 may be repositioned. This may be repeated until an optimal result is produced wherein the leaflets LF are held in place.

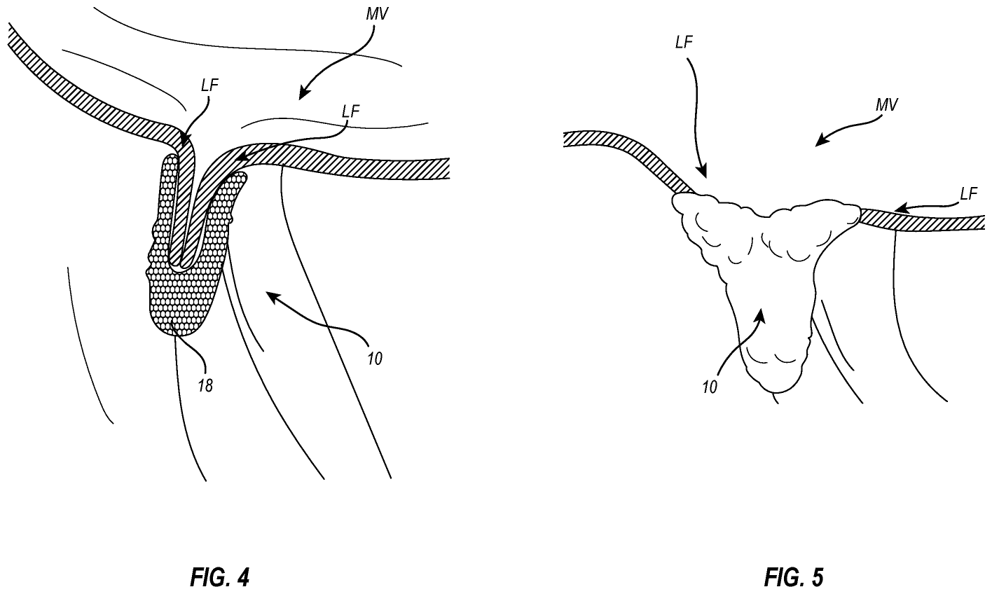

FIGS. 4 and 5 illustrate the fixation device 10 once deployed. As illustrated, the fixation device 10 may optionally include a covering, such as covering 16. The covering 16 may assist in grasping the tissue and may later provide a surface for tissue ingrowth, such as illustrated in FIG. 5. Ingrowth of the surrounding tissues, such as the valve leaflets, provides stability to the device 10 as it is further anchored in place and may cover the device with native tissue, thus reducing the possibility of immunologic reactions. The covering 16 may be comprised of any biocompatible material, such as polyethylene terephthalate, polyester, cotton, polyurethane, expanded polytetrafluoroethylene (ePTFE), silicone, or various polymers or fibers and have any suitable form, such as a fabric, mesh, textured weave, felt, looped or porous structure. Generally, the covering has a low profile so as not to interfere with delivery through an introducer sheath or with grasping and coapting of leaflets or tissue. Additional description regarding such coverings may be found in PCT Publication No. WO 2004/103162, the disclosure of which is incorporated herein by reference in its entirety.

### III. Methods of Removing a Fixation Device

Sometimes, after installation of a fixation device in the heart, it needs to be removed. Ordinarily, this has been done during an invasive procedure such as open-heart surgery. Invasive procedures such as these often have high risk of complications, however. Further, sometimes mitral valve fixation devices are installed on patients for whom open heart or more invasive procedures are otherwise unnecessary or undesirable. For these patients, and even for patients in whom open-heart surgery is used, it would be beneficial to have devices and systems specifically designed for removing the fixation devices within an endovascular procedure, rather than a procedure requiring open heart access.

Minimally invasive systems, methods, and devices for removing the fixation devices are disclosed herein. These minimally invasive systems, methods, and devices allow a practitioner to remove the fixation device and, optionally, then proceed to do other things in the heart, without necessarily requiring open heart access or other more invasive procedures. Such systems, methods, and devices are configured to be effective even if the fixation device has been installed for weeks, months, or years, such that tissue surrounding the device may have grown over, into, or around the fixation device. As a result of such tissue ingrowth, or for other reasons, removal of the fixation device as described above that may be practical during the initial placement procedure may no longer be practical. The systems, methods, and devices disclosed herein may also be useful for adjusting the installation of a mitral valve fixation device after it is installed.

An embodiment of the present invention discloses systems that include various devices that may include catheters and tools that perform various functions, and also multifunctional catheters and tools that can perform any combination of functions. Such functions may include holding or retaining an installed fixation device; cutting or otherwise partitioning a leaflet or leaflets; removing a fixation device; and subsequently repairing the leaflet(s) or associated valve. Related methods for performing such functions are also disclosed.

The devices and associated methods and systems described herein may be used in combination with imaging modalities such as x-ray, fluoroscopy, echocardiography, charge coupled device cameras, complementary metal oxide semiconductor cameras, magnetic resonance imaging, and other imaging modalities. The availability of such imaging modalities during such procedures may help practitioners visualize, for example, where the fixation devices are, how they are connected to the heart, and where to direct the various catheters and/or other devices.

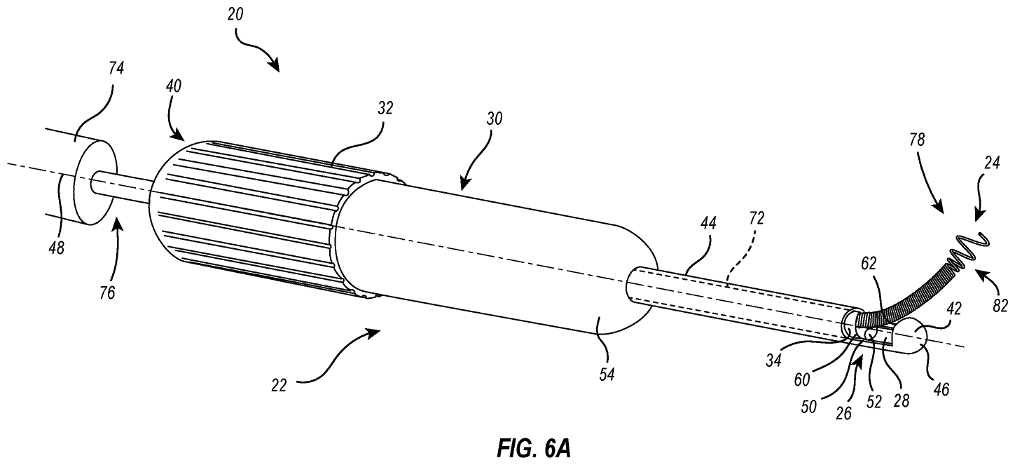

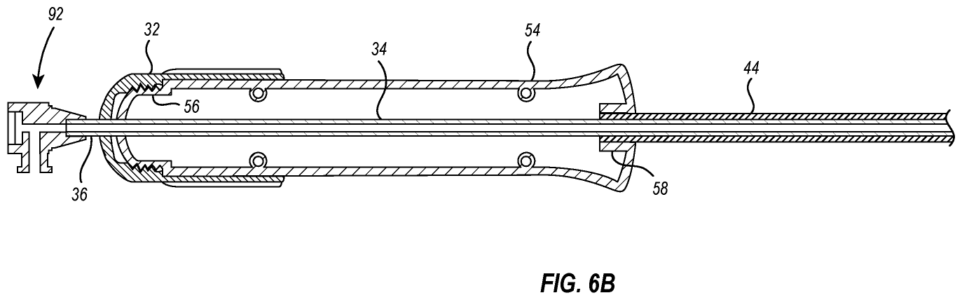

Turning to FIG. 6A illustrated is an implant removal system 20 that can be used to capture and subsequently remove a fixation device, such as the fixation device 10 of FIGS. 3-5. The implant removal system 20 can be used to access the fixation device within a heart cavity, such as when the fixation device 10 captures mitral valve leaflets (LF). This can be achieved through a transapical antegrade approach or transfemoral retrograde approach. While the transapical approach will be discussed in more detail hereinafter, it will be understood that the implant removal system 20 can also be used for a transfemoral approach through lengthening and increasing a flexibility of various components, providing steering capabilities, and making such other modifications that one-skilled in the art can contemplate based upon the underlying disclosure presented herein.

As illustrated in FIG. 6A, the implant removal system 20 includes an implant management tool 22 and an implant removal device 24 that can be deployed from the implant management tool 22 to position an end of the implant removal device 24 in close proximity to a previously deployed fixation device, such as fixation device 10 (FIGS. 3-5). In the presently illustrated configuration, the implant removal device 24 can be used to capture the fixation device 10 (FIGS. 3-5), including some ingrowth tissue, leaflet tissue or other tissue (collectively referred to herein as “tissue”), while the implant management tool 22 can be used to separate the captured fixation device 10 (FIGS. 3-5) and tissue from the surrounding anatomy. For instance, once the implant removal device 24 captures the fixation device 10 and tissue, and the captured fixation device 10 and tissue are withdrawn through an opening 26 into a region 28 of the implant management tool 22, manipulation of an actuator 32 of a handle 30 can move a cutting member 34 to at least partially close the opening 26 and cut the tissue surrounding the fixation device 10 (FIGS. 3-5). The detached fixation device 10 (FIGS. 3-5) is retained within the region 28 formed at the opening 26, that is at least partially closed by the cutting member 34. Once the fixation device 10 (FIGS. 3-5) is detached from the surrounding tissue, the implant removal system 20, with the captured fixation device 10 (FIGS. 3-5), can be removed from the patient. Thereafter, the leaflet(s) are repaired or an artificial or replacement valve is deployed, such as taught in U.S. Pat. Nos. 9,895,221 and 10,6175,519 and U.S. Patent Publication No. 2018/0078370, the disclosures of which are incorporate herein by this reference.

The implant management tool 22 has a proximal end 40 and a distal end 42, with the handle 30 disposed towards the proximal end 40, with a shaft 44 extending from a handle distal end towards the distal end 42 of the implant management tool 22. The distal end 42 has a curved outer surface 46, as an atraumatic surface or tip, to reduce tissue damage when the implant management tool 22 is advanced through tissue associated with an apical, transfemoral, trans-jugular, trans-radial, or other approach. The opening 26 toward the distal end 42 opens transversely to a longitudinal axis 48 of the shaft 44 so the fixation device 10 (FIGS. 3-5) and tissue can be received through a side 50 of the shaft 44. With the opening 26 being elongate along a longitudinal axis 48 of the shaft 44, a greater space can be provided to capture the fixation device 10 (FIGS. 3-5) and tissue than if the opening 26 were, for example, coaxial with the lumen 52 of the shaft 44. However, having the opening 26 coaxial with the lumen 52 or having the opening 26 opening from the distal end 42 of the shaft 44 in a direction parallel to a longitudinal axis 48 of the shaft 44 is alternatively possible and consistent with the teaching presented herein, as will be discussed hereinafter.

The handle 30 includes a cutting member actuator 32 rotatably mounted to a handle body 54 so that rotational movement of the cutting member actuator 32 in one direction translates or slides the cutting member 34 to at least partially close the opening 26 formed at the distal end 42 and rotational movement in an opposite direction translates or slides the cutting member 34 to at least partially open the opening 26. This translating or sliding movement can begin with a cutting member distal end 42 partially disposed within the region 28, as shown in FIG. 6A, or alternatively the cutting member distal end 42 can be fully retained within a lumen 52 of the shaft 44 prior to positioning the fixation device 10 (FIGS. 3-5) and tissue within the region 28. In the latter case, the space available to accommodate the fixation device 10 (FIGS. 3-5) and tissue is increased as compared to when the cutting member distal end 42 partially extends into the region 28. Optionally, moving the cutting member actuator 32 not only translates or slides the cutting member 34 but also rotates the cutting member 34 to aid with cutting or shearing tissue. Such rotation can include greater or lesser than 360 degrees of rotation.

The cutting member 34 is a generally tubular member with a cutting edge 60 at a cutting member distal end 42 and a luer port 92 at a cutting member proximal end 36 as illustrated in FIG. 6B; the luer port 92 preventing air ingress through use of a hemostatic valve, such as a rotating hemostatic valve, but allowing guidewires and snares to be inserted and translated without air ingress or loss of blood. The cutting edge 60, and so the cutting member distal end 42, can be at least partially received within a distal recess 62 formed near the distal end 42 following manipulation of the cutting member actuator 32 to translate or slide, and optionally rotating, the cutting member 34 to at least partially close the opening 26 formed at the distal end 42. The distal recess 62 accommodates the cutting edge 60 so that an end wall 64 of the shaft 44 and the cutting edge 60 can provide a scissor-like cutting action to cut the tissue. To achieve this scissor-like cutting action the cutting edge 60 and/or the end wall 64 can be sharpened along their perimeter or can be serrated or include other low and high points to aid with cutting the tissue.

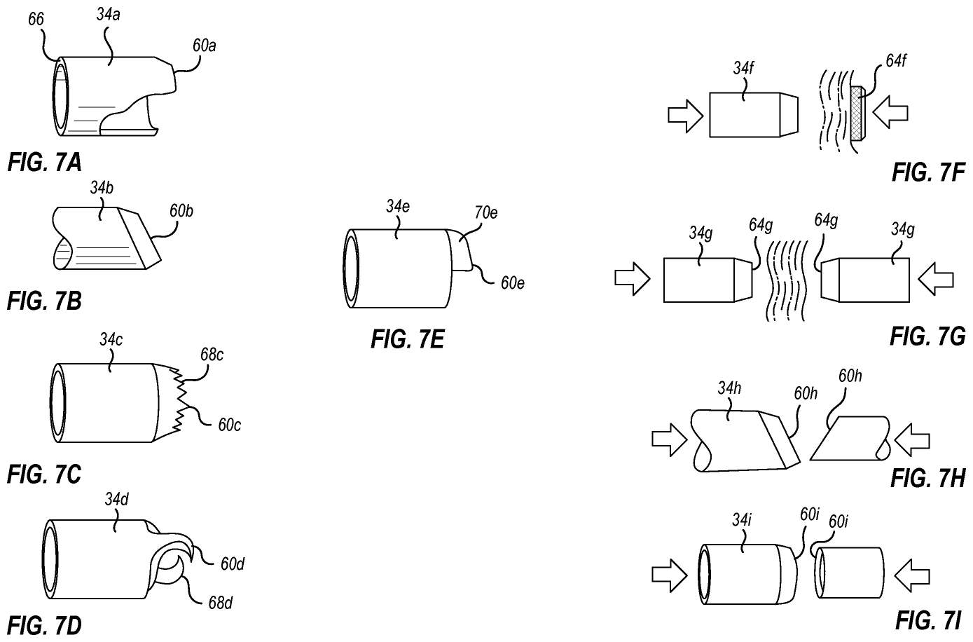

While a general discussion of the cutting member is presented above, referring now to FIGS. 7A thru 7I, exemplary embodiments of the cutting member are illustrated. For ease of explanation, the illustrated cutting members are depicted in relation to tissue without a fixation device and associated tissue. It will be understood, however, that the illustrated cutting members can be used to remove the fixation device and tissue by disposing the same with a lumen of the cutting member as discussed herein.

Referring now to FIG. 7A, the cutting member 34a resembles a biopsy punch, where the cutting member 34a includes a hollow thin-walled metal cylinder 66 in which the cutting edge 60a is formed, such as a razor-like cutting edge. It should be appreciated that the cutting member 34a can have different circumferential shapes including, but not limited to, a circle, an ellipse, a rectangle, a rectangle with rounded corners, etc. The cutting edge 60a can meet the cylindrical wall perpendicularly, or at an angle, as illustrated as the cutting edge 60b of cutting member 34b (FIG. 7B).

Referring now to FIG. 7C, a cutting edge 60c of a cylindrical cutting member 34c may be serrated. The serrations 68c can be uniformly or non-uniformly distributed or arranged in a particular manner in which some serrations can optionally be of a different size and shape than others. The serrations 68c can assume the typical triangular shape, or can take other more exotic shapes like that of a sickle 68d (FIG. 7D), spade, or the like. Additionally, while each of the cutting edges can be annular so that it approximates a tubular shape, it will be understood that the sharpened portion of the cutting edge 60 and/or the end wall 64 can be formed by an arc portion 70e of a complete annular edge of the cutting edge 60e, such as illustrated in FIG. 7E.

The cutting member 34 described herein can have one or both of two types of movements: the first being a translation along the longitudinal axis of the cutting member 34 in the direction that moves the cutting edge toward tissue to be excised; and the second being rotation of the cylindrical cutting member 34 about its longitudinal axis. In the illustrated configuration of FIG. 6A, the cutting member 34 is both translated along the longitudinal axis and rotated about its longitudinal axis so that the cutting edge 60 of the cutting member 34 shears the tissue such as by imparting on the target tissue both a pushing and a sliding motion, such as when cutting member actuator 32 is manipulated by a user. The cutting member 34 in this embodiment will have a circular circumferential shape to allow for the desired rotational and longitudinal movement. This movement can be achieved, in one configuration, with a proximal end of the cutting member 34 mounted to a proximal end of the cutting member actuator 32, such as illustrated in FIG. 6B. Rotation of the cutting member actuator 32 about a threaded portion 56 of the handle body 54, the cutting member actuator 32 having a complementary threaded portion 38, allows the cutting member 34 to translate and rotate in relation to the stationary shaft 44 that is mounted to a mounting surface 58 of the handle body 54. The engagement between the threaded portion 56 and the complementary threaded portion 38 provides a safeguard on the cutting feature to be controlled and steady, so as not to cause unintended movements with a sharp edge to cause harm). Additionally, the threaded portion engagement provides a mechanical advantage to allow for small motions resulting in greater cutting/shearing force on the target tissue.

The cutting member 34 may be the only element of a cutting arrangement, and the one blade is advanced towards the desired tissue throughout the duration of the cutting process. For instance, the cutting member 34f is advanced toward the end wall 64f with the end wall 64f functioning like a backstop, cutting mat, or anvil for the cutting member 34f to press against, as illustrated in FIG. 7F. With the tissue resting against the end wall 64f, the end wall 64f creates a more stable cutting configuration.

Alternatively, a cutting arrangement of two cutting members may replace the single cutting member, as illustrated in FIGS. 7G-7I. This can be the case, as described above, when the cutting member 34 is at least partially received within the distal recess 62 (FIGS. 6A-B) and cooperates with the end wall 64 (FIG. 6A-B) to cut the tissue. As shown in FIG. 7G, and described above when the end wall 64g is sharpened to include the cutting edge 60g, the two cutting members 34g can work together acting like jaws, where a target tissue is first positioned between two cylindrical cutting member 34g having their cutting edges 60g facing each other. Either one or both of the cutting member 34g can be advanced toward the other until the target tissue is completely cut through. It should be appreciated that the cutting member 34g can be made such that one is smaller and can be nestled concentrically in the other, as shown in FIGS. 7F, 7H, and 7I, insuring that the cutting edges 60f, 60h, 60i can move past each other for a more effective overall cutting motion. In any one of the aforementioned embodiments, the blade or blades can also rotate about their longitudinal axes to impart a sliding motion to the cut.

It should be appreciated that a radiofrequency (RF) or an ultrasonic cutting arrangement can be used instead of the cutting member blade. The shape of the RF cutting element or the ultrasonic cutting element can adopt any of the above-described configurations.

Returning to FIG. 6A, the implant removal device 24 extends from the proximal end 40, through the handle 30, the shaft 44, and the cutting member 34 to exit the opening 26 at the distal end 42. The implant removal device 24 extends through a lumen 72 of the shaft 44 and the lumen 52 of the cutting member 34 and can be slid, translated, and/or rotated along and within the lumens 52, 72 to position an end of the implant removal device 24 in close proximity to a previously deployed fixation device 10 (FIGS. 3-5). This sliding, translating, and/or rotating action can be achieved through connecting a torque handle 74 to an implant removal device proximal end 76. The torque handle 30 allows a user to manipulate the implant removal device 24 to position an implant removal device distal end 78 in close proximity to a previously deployed fixation device 10 (FIGS. 3-5). For instance, the implant removal device 24 is resiliently flexible and configured to transmit torque from the implant removal device proximal end 76 to the implant removal device distal end 78 with a predetermined torque or turning ratio, i.e., ratio of implant removal device proximal end 76 rotation to the implant removal device distal end 78 rotation. This predetermined torque or turning ratio is 1:1, but can range from 3:1 to 2:1, from 1:2 to 1:3. (proximal turns:distal turns)

The implant removal device 24 includes an elongate body 80 with a capture structure 82 extending from an elongate body distal end. The torque handle 74 can selectively mount to an elongate body proximal end of the elongate body 80 at the implant removable device proximal end 76; the elongate body 80 being resiliently flexible and configured to transmit torque from the elongate body proximal end to the elongate body distal end so that rotational movement of the elongate body proximal end translates to rotation movement of the elongate distal end, including the capture structure 82 at the elongate distal end 42. By so doing, this allows a user to accurately position the capture structure 82 during fixation device capture and removal.

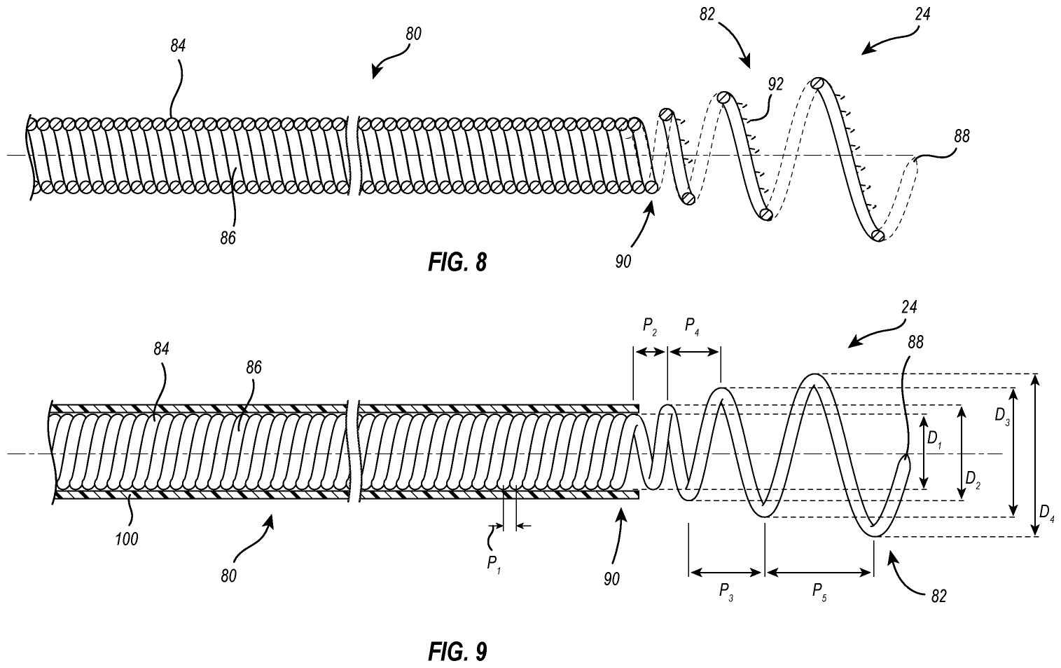

Turning to FIGS. 8 and 9 illustrated are configurations of the implant removal device 80, with FIG. 9 illustrating the implant removal device with an optional sheath 100, which will be discussed in more detail hereinafter. As illustrated, the implant removal device 24 includes a coiled member 84 that forms both the elongate body 80 and the capture structure 82. The coiled member 84 forms a plurality of coils 86 that coil in one direction, such as clockwise or counter-clockwise, with those coils 86 having a constant pitch P1 along a length of the elongate body 80. The coiled member 84 is flexible in bending and provides little or no elongation in either compression or tension. In one configuration, for instance, the coiled member 84 along the elongate body 80 is a stacked coil, where adjacent coils 86 contact each other, along the length of the elongate body 80. With adjacent coils contacting each other, there is no elongation in compression of the elongate body 80 during distal movement. In tension, such as when the elongate body 80 is withdrawn proximally, the coils 86 remain substantially in contact with each other. Alternatively, in tension there can be a small amount of separation so that any elongation of the elongate body 80 is between about 5 percent and about 30 percent of the length of the elongate body. With such elongation, a physician can manipulate and position the implant removal device 24 with the patient's anatomy to capture the fixation device 10 (FIGS. 3-5) and tissue.

While reference is made to use of a stacked coil, it will be understood that the coils 86 of the elongate body 80 can have some separation between the coils 86 while maintaining the desired torque transmission. This separation can by about 0.1 mm to about 0.5 mm, from about 0.5 mm to about 1.0 mm, from about 1.0 mm to about 1.5 mm, or from about 1.0 mm to about 2.0 mm. With such configurations, the coiled member 84 can elongate in length by about 0 percent and about 2 percent, about 2 percent and about 5 percent, about 5 percent and about 10 percent, about 10 percent and about 15 percent, or about 5 percent and about 30 percent.

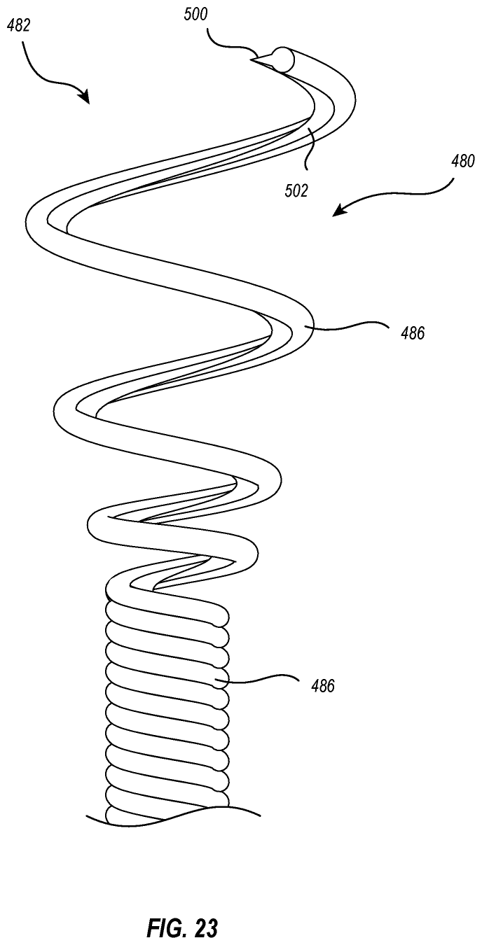

In contrast to elongate body 80, a portion of the coiled member 84 forming the capture structure 82 has a constant or variable pitch and a terminal tip end or tip 88. A diameter of the coils 86 increase in diameter towards the implant remove device distal end 78 to provide a generally conical or trumpet-shape with a capture opening 26 that can receive an end of the fixation device 10 (FIGS. 3-5) and tissue. With the capture opening 26, as the elongate body 80 is advanced toward the fixation device 10 (FIGS. 3-5), the capture structure 82 can guide the fixation device 10 (FIGS. 3-5) within the capture opening 26 so that a longitudinal axis of the fixation device 10 (FIGS. 3-5) becomes generally aligned with a longitudinal axis of the elongate body 80. Such guiding can include one or more of compressing, cutting, and penetrating the tissue. For instance, rotating the implant removal device 24 can advance the tip 88, and optionally other cutting surfaces (see FIG. 23) of the capture structure 82, so that the tissue is compressed by the capture structure 82. Additionally, rotating the implant removal device 24 can also cut and penetrate the tissue. This screw-type action of the capture structure 82, whether or not there is actual penetration or cutting of the tissue, can draw the fixation device 10 (FIGS. 3-5) into the capture opening 26 and securely capture and center or self-center the fixation device 10 (FIGS. 3-5) and associated tissue, within the capture opening 26. A fixation device that is not coaxial with the elongate body 80 and/or the capture structure 82 can be simply retrieved and positioned to approximate coaxial alignment with the elongate body 80 so the fixation device and tissue can be withdrawn through the opening 26 and into the region 28 of the implant management tool 22 more efficiently. Stated another way, the capture structure is configured to selectively center a deployed implant, such as the fixation device, in relation to a longitudinal axis of the elongate body and the capture region to aid with capture and subsequent removal of the fixation device.

A resiliency of the capture structure 82 provides a compressive force upon the fixation device and the tissue in a direction transverse to the longitudinal axis of the capture structure 82. So even if a tip 88 of the capture structure 82 does not cut or penetrate the tissue during the screw-type action, the coils 86 of the capture structure 82 apply a sufficient compressive force to securely hold the fixation device 10 (FIGS. 3-5) and the tissue within the capture opening 26. The compressive force can range from about 0.9 Newtons to about 1.3 Newtons, from about 1.1 Newtons to about 1.5 Newtons, from about 2.2 Newtons to about 4.4 Newtons, from about 4.4 Newtons to about 6.6 Newtons.

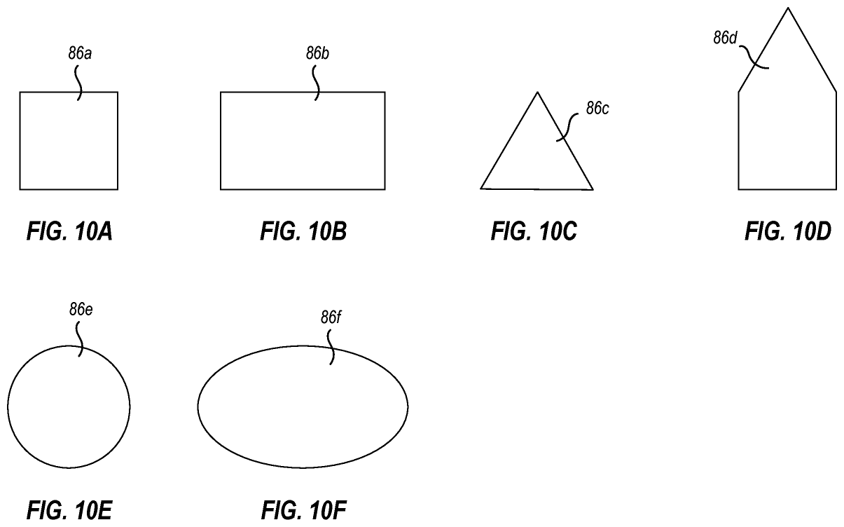

To also aid with capture of the fixation device 10 (FIGS. 3-5) and tissue retention, the coils 86 can have a cross-sectional profile to increase contact surfaces between the coils 86 and the tissue. While a generally circular coil cross-section can be used, a polygonal cross-section or inclusion of features to aid frictional engagement between the coils 86 and the fixation device 10 (FIGS. 3-5) and tissue are possible. For instance, and not by way of limitation, as illustrated in FIGS. 10A-10F, the coils 86 can have cross-sections that are square, rectangular, triangular, polygonal, oval, elliptical, combinations or modifications thereof. It will also be understood that for those cross-sections having a major axis and a minor axis, either the major axis or the minor axis can be disposed to be generally parallel to or transverse to a longitudinal axis of the capture structure 82 or the elongate body 80. Changing the orientation of the major axis and the minor axis can be used to vary the flexibility of the capture structure 82 or the elongate body 80. With respect to features to increase frictional engagement, this can include projections, barbs, roughened surface finishes, coatings, rotational bias (ratcheted edges that allow for smooth rotation in the direction of the coil wind that engage the tissue when rotating in the counter direction of the coil wind—e.g. smooth clockwise rotation but tissue engaging counter-clockwise), combinations or modifications thereof.

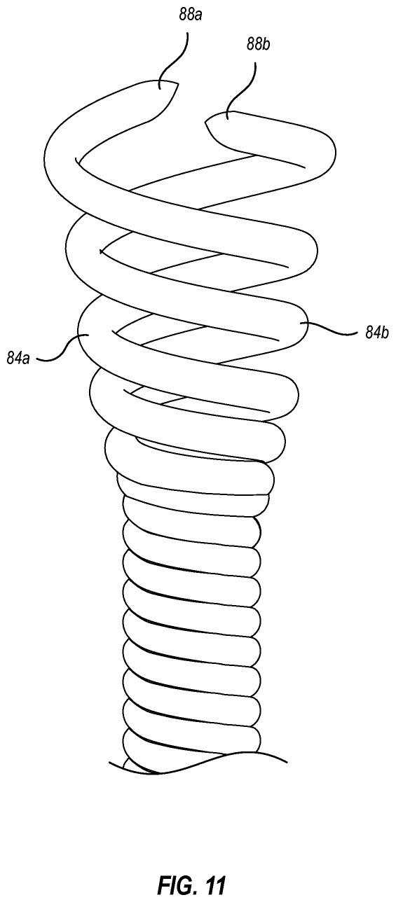

With continued reference to FIGS. 8 and 9, the coiled member 84 is a single coil with a single tip 88. However, the coiled member 84 can include multiple coil members and/or tips, such as a double spiral with two coiled members 84a, 84b and two tips 88a, 88b, such as illustrated in FIG. 11. More generally, the coiled member 84 can have one or more coil members and one or more tips.

As illustrated in FIGS. 8 and 9, a pitch of the coils 86 in the capture structure 82 continually increase towards a capture structure distal end 42, corresponding to the elongate body distal end 42. A pitch of the coils in the capture structure 82 portion formed by the coiled member 84 are greater than a pitch of the coils of the elongate body 80 portion. For instance, the coils 86 of the coiled member 84 of the elongate body 80 portion are in contact with each other as a stacked coil having a pitch P1. In contrast, the coils 86 of the capture structure 82 are illustrated as having increasing pitch from a transition or junction 90 between the capture structure 82 and the coiled member 84 toward the distal end 42 of the implant removal device 24. For instance, a pitch P1 is smaller than a pitch P2, the pitch P2 is small than a pitch P3, the pitch P3 is small than a pitch P4, and the pitch P4 is small than a pitch P5.

A diameter of the capture structure 82 increases along a length of the capture structure 82. For instance, a diameter D1 may smoothly transition from an outer diameter of elongate body 80 (with or without a coating or liner), and increase to the wider end diameter D4. The increasing taper shape of capture structure 82 may be a linearly increasing ratio (example: increasing 10% in diameter for every 1 mm of increasing length, but not to exceed the final D4). The diameter D4 should not exceed the inner diameter of a lumen of the sheath 100, which will be discussed in more detail hereinafter. The increasing taper shape may be non-linear, increasing at a greater ratio, e.g. 20%, 25%, 30% or some ratio or decreasing at a lesser ratio, e.g., between about 1%-10%, for a given length, and a reduced ratio for the remaining length, e.g. 5%, 4%, 3%, 2%, 1% or some other ratio less than 10%). The particular tapering map can be optimal for positioning and redirecting the distal tip of the implant into the center of the tapered capture structure. Likewise, a relative flexibility may conform to the trajectory of the implant axis if not colinear with the axis of the capture structure—aided by the engaging features of the capture structure, and by additional rotation and torque to aid in drawing the implant and tissue into the capture structure.

In some configurations, the diameter D4 can range from about 4 mm to about 12 mm, from about 4 mm to about 10 mm, from about 3 mm to about 9 mm, or from about 2.5 mm to about 8 mm, with the other diameter ranges D1-D3 being based upon the tapered ratios discussed above.

Additionally, the diameters of the capture structure 82 can vary based upon an estimated size of the fixation device and any tissue to be removed with the fixation device. For instance, in one procedure a physician can measure the ingrown fixation device using fluoroscopy, intracardiac echocardiogram (ICE), three-dimensional (3D) electroanatomical mapping (EAM) systems, or other imaging modalities and select a particularly sized implant removal device. Alternatively, a single implant removal device can accommodate various sizes of ingrowth fixation device, with associated tissue, with the physician using the screw-type action to cut, penetrate, or otherwise capture the ingrowth fixation device.

While the illustrated configuration includes both increasing pitch and coil diameter so that the capture structure 82 has a generally conical shape, it will be understood that in other configurations, the coils 86 can have (i) a constant pitch with increasing diameter over a length of the capture structure 82, (ii) a constant pitch with constant diameter over a length of the capture structure 82 resulting in a generally cylindrical shape, (iii) an increasing pitch with increasing diameter over a length of the capture structure 82, or (iv) combinations thereof or modification thereto.

Generally, the implant removal device 24 can be fabricated from various materials, such as metals, alloys, polymers, ceramics, shape memory materials including shape member alloys or shape member polymers, superelastic materials, combinations thereof or modifications thereto. Additionally, the material forming the implant removable device 24 can be processed to achieve differing flexibilities along the length of the implant removal device 24. For instance, the junction 90 between the elongate body 80 and the capture structure 82 can be cold worked to increase or decrease the stiffness to change the flexibility characteristics or properties. Providing decreased stiffness, increasing flexibility, allows the capture structure 82 to locate the fixation device more easily. Instead of cold working, it will be understood that adjacent coils can be welded or otherwise bonded together, thereby increasing a stiffness of the implant removal device 24 at the location of the welding or bonding.

While discussion is made to the elongate body 80 being a stacked coil, in other configurations, the elongate body 80 can be a hypotube, a braided tubular member, a composite where sections or lengths of coils may be constrained by a heat-shrink laminate or polymer filling interstitial spaces between coils or inner/outer or both laminated layers to prevent elongation or increase bi-directional torsion combinations or modifications thereof, or other structure where the elongate body is a solid member or includes a elongate body lumen 52.

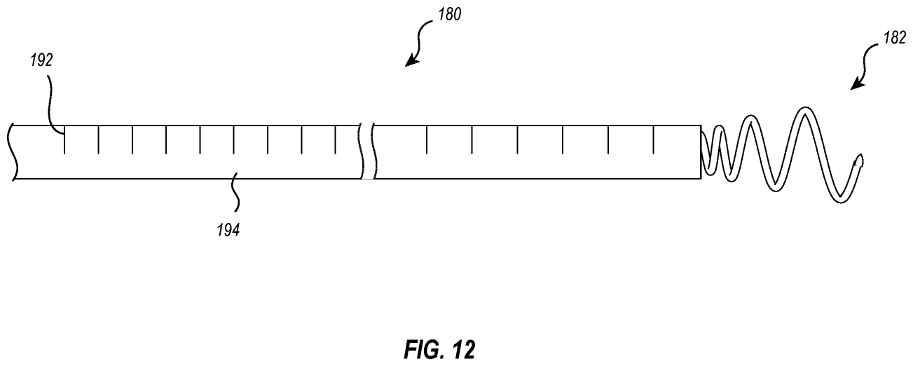

As illustrated in FIG. 12, an alternate configuration of the implant removal device is illustrated. For simplicity, like reference numerals are associated with like structures and the discussion of other implant removal devices herein is also applicable to this configuration. When the elongate body is a hypotube, the elongate body 180 can include a plurality cuts or slits 182 that control a flexibility of the elongate body 180. The cuts or slits 182 can be elongate, form through holes or island cuts, combinations or modifications thereof. The cuts or slits 182 can be linear, diamond-shaped, square, rhombohedral, triangular, rectangular, circular, oblong, other elliptical or oval shapes, other regular or irregular polygonal shapes, or modification or combinations thereof.

The cuts or slits 182 can form at least one longitudinally continuous spine 194 that can preferably be continuous and uninterrupted along a longitudinal length of, and located at a fixed angular location on, the elongate body 180. Having a longitudinally continuous spine 194 allows the elongate body 180 to transmit tension force applied at the elongate body proximal end to the elongate body distal end without substantial elongation of the elongate body 180. In other embodiments, the longitudinally continuous spine 194 can may allow the elongate body 180 to transmit compression force applied at the elongate body proximal end to the elongate body distal end without substantial shortening of the elongate body. For example, some embodiments of an elongate body can exhibit a change in a longitudinal length of less than 30% during application of a compression force of 4 Newtons or greater and/or application of a tension force of 4 Newtons or greater.

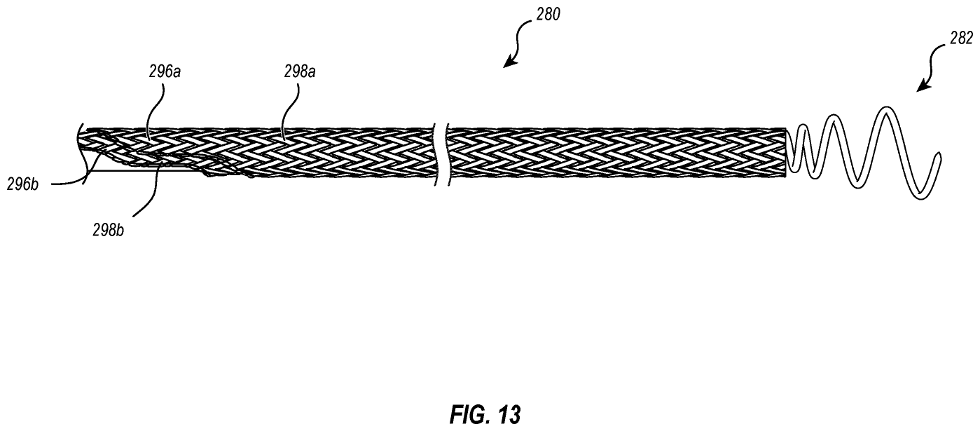

As illustrated in FIG. 13, an alternate configuration of the implant removal device is illustrated. For simplicity, like reference numerals are associated with like structures and the discussion of other implant removal devices herein is also applicable to this configuration. When the elongate body is a braided tubular structure 280 it can include a braid configuration that prevents unwanted compression during distal translation and unwanted elongation under tension during proximal translation of the elongate body 180 during positioning of the capture structure 282. For instance, the braid can include a plurality of threads that are woven together to provide one or more layers 296. For example, a layer 296a may include one pattern of threads 298a, while the layer 296b has a different or alternate pattern of threads 298b. The threads 298a, 298b can extend at various angle to one another and can be woven together in a repeating pattern. For instance, either or both of the threads 298a, 298b may be woven in a diamond two wire two-under-two, over-two pattern; a half-load single wire over-one, one-under pattern; a full-load single wire over-two, under-two pattern; other alternating woven patterns; or combinations thereof. In other embodiments, braid can include a single thread routed substantially straight longitudinally through the plurality of threads.

The threads 298a, 298b can be round threads, elliptical threads, or flat threads. The threads 298a, 298b can be made of or include a variety of reinforcement materials, such as, metals, metal alloys, thermoplastics, other polymers, or combinations thereof. In some embodiments, the reinforcement material or materials may have a greater elastic modulus than the body material. For example, the braid can include a mixture of threads 298a, 298b with different properties, such as metal or stainless-steel threads woven with polymer threads. In at least one embodiment, the braid can include a plurality of 304 stainless-steel wires woven in a diamond pattern. Such an embodiment of the braid can include between 16 and 32 threads of stainless steel.

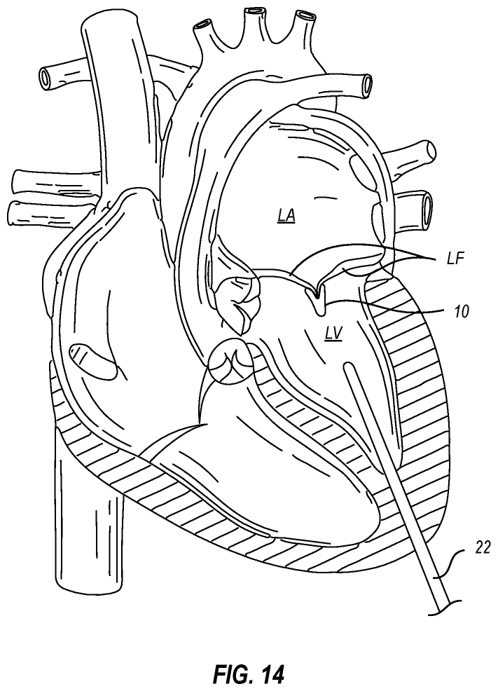

Referring now to FIG. 14 is a cut-away view of a heart with a portion of an implant management tool 22 according to the present invention and accessing the heart using an apical approach. It is contemplated that other surgical approaches to the heart, and valves in addition to the mitral valve, are within the scope of the inventive subject matter claimed herein. FIG. 14 shows a portion of the implant management tool 22 advanced toward mitral valve having a previously deployed fixation device, such as the fixation device 10 (FIGS. 3-5).

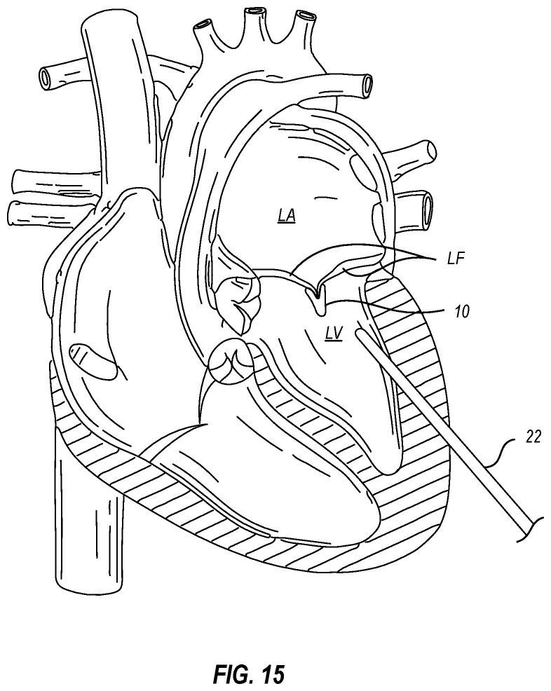

Referring now to FIG. 15, FIG. 15 shows the lateral deployment of one embodiment of portion of an implant management tool 22 according to the present invention and shows a portion of an implant management tool positioned within the left ventricle towards the mitral valve by way of a lateral trans-ventricular wall approach through the lateral wall of the left ventricle of the heart.

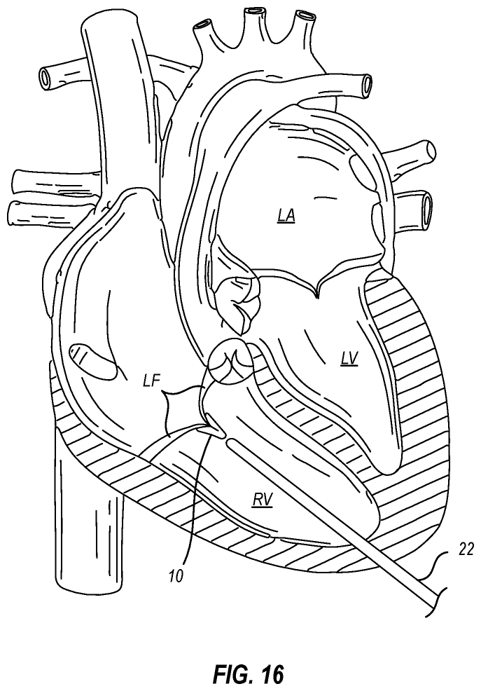

Referring now to FIG. 16, FIG. 16 is a cut-away view of a heart with a portion of an implant management tool 22 according to the present invention and accessing the heart using an apical approach into the right ventricle. It is contemplated that other surgical approaches to the heart, and valves in addition to the mitral valve, are within the scope of the inventive subject matter claimed herein. FIG. 16 shows the portion of an implant management tool advanced toward the tricuspid valve.

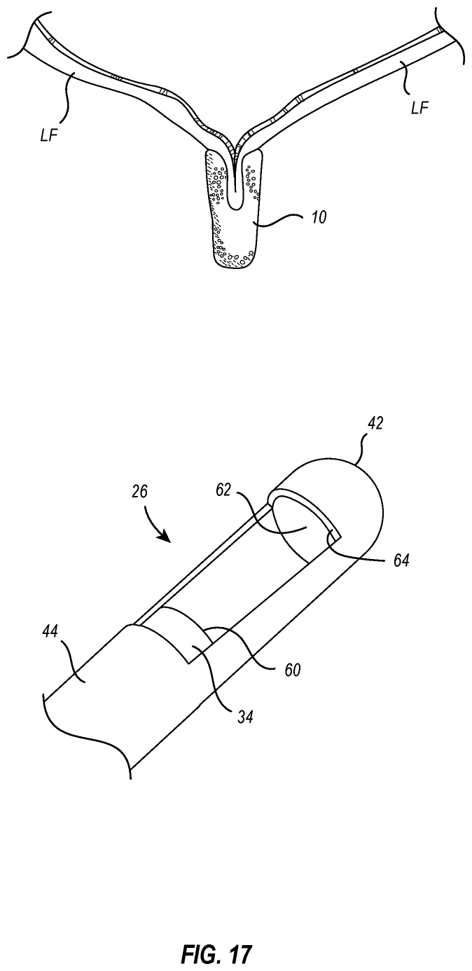

FIGS. 17-21 is a series of drawings illustrating the deployment of the implant management tool and the implant removal device and subsequent capture and removal of a previously deployed fixation device, such as the fixation device 10 (FIGS. 3-5). For simplicity, FIGS. 17-21 illustrate a portion of the anatomy from FIGS. 14-16 and a portion of each of the implant management tool and the implant removal device.

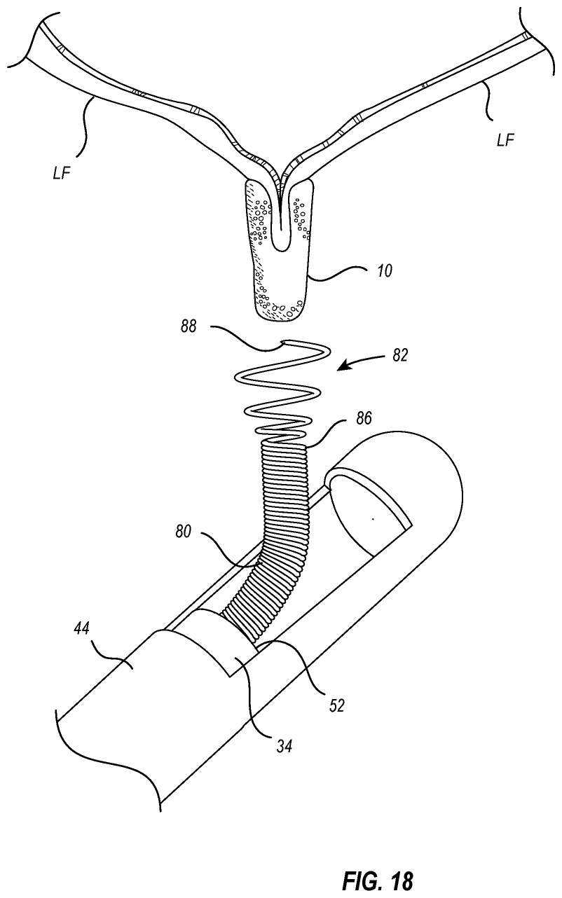

As illustrated in FIG. 17, the implant management tool 22 is advanced toward the previously deployed fixation device 10. With the opening 26 being generally orientated toward the deployed fixation device 10, the implant removal tool 24 can be translated or moved along the lumen 52 so the capture structure 82 is advanced toward the deployed fixation device 10, as illustrated in FIG. 18. This can be achieved to by manipulating the proximal end 40 of the implant removal tool 24 either directly or by way of the torque handle 74.

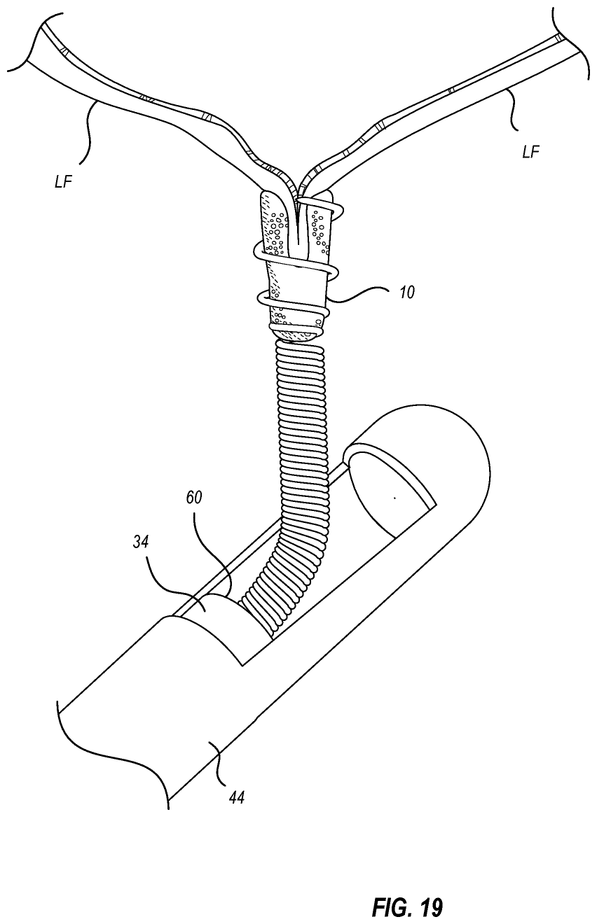

A user can further manipulate the proximal end 40 to advance the capture structure 82 over the fixation device 10, as illustrated in FIG. 19. As the user advances the capture structure 82 towards an end of the fixation device 10, such as by torqueing the elongate body 80 to rotate the capture structure 82 and advance the capture structure 82 along a portion of tissue surrounding the fixation device 10, the conical shape of the capture structure 82 begins to axially align the fixation device 10 with a longitudinal axis of the elongate body 80. For instance, as the implant removal tool 24 is rotated, the coils 86 engage with the tissue, applying a compressive force against the tissue and the fixation device 10 and draw the fixation device 10 and tissue into the capture opening 26. Optionally, the tip 88 can penetrate the tissue so the coils 86 penetrate the tissue, to provide enhanced capturing of the previously deployed fixation device 10 through both compressive force and mechanical engagement with the tissue.

When the capture structure 82 has been advanced sufficiently along the length of the fixation device 10, such that the last coil of the capture structure 82 is positioned at close to or past an end of the fixation device 10 closest to the leaflets, a physician or clinician can verify its location by fluoroscopy, intracardiac echocardiogram (ICE), three-dimensional (3D) electroanatomical mapping (EAM) systems, or other imaging modalities. Because the capture structure 82 is formed of a radiopaque material, the physician or clinician can verify the location with relative ease.

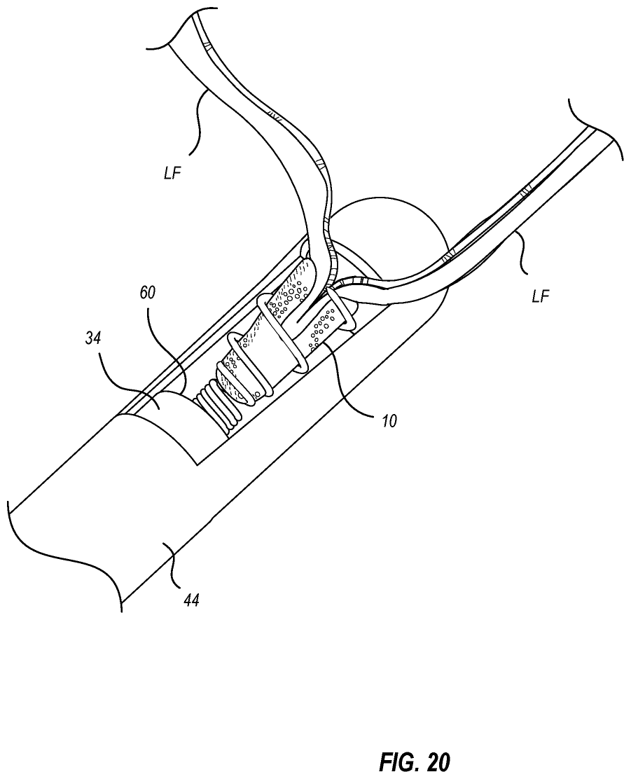

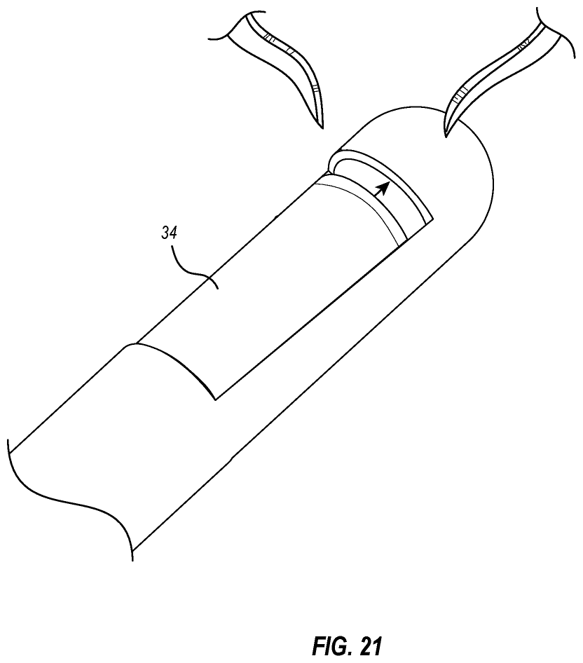

Once verified, the implant removal device 24 is drawn proximally to apply tension to the capture structure 82, as illustrated in FIG. 20. This tension can at least partially increase a length of the capture structure 82, including increasing a pitch of the coils of the capture structure 82, and increase a compressive force on the fixation device 10 and the tissue. The compressive force can be a transverse force that is applied transversely to a longitudinal axis of the capture structure 82.

With the tissue partially taught, manipulation of the actuator 32 of the handle 30 can translate, and optionally rotate, the cutting member 34 to at least partially close the opening 26 and cut or separate the fixation device 10 from the tissue surrounding the fixation device 10, as illustrated in FIG. 21. For instance, the actuator 32 advances the cutting member 34 with an annular cutting edge to cut the tissue. The detached fixation device 10 is retained within the region 28 formed at the opening 26, that is at least partially closed by the cutting member 34. Once the fixation device 10 is detached from the surrounding tissue, the implant removal system 20, with the captured fixation device 10 can be removed from the patient.

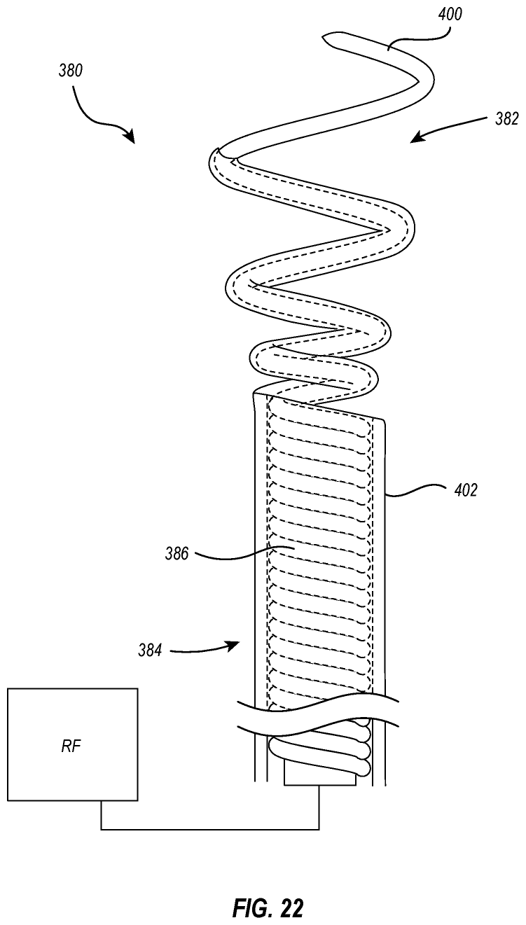

Turning to FIG. 22, illustrated is an alternate embodiment of the capture structure according to the present invention. For simplicity, like reference numerals are associated with like structures and the discussion of other implant removal devices herein is also applicable to this configuration. As mentioned previously, a radiofrequency (RF) or an ultrasonic cutting arrangement can be used instead of the cutting member. The shape of the RF cutting element or the ultrasonic cutting element can adopt any of the above-described configurations.

In the illustrated configuration of FIG. 22, the implant removal device 380 is configured to include an RF cutting arrangement, thereby eliminating the need for the cutting member of the implant management tool and, in some configurations, the implant management tool as a whole. For instance, a catheter can be used to access the previously deployed fixation device, such as using an apical, transfemoral, trans-jugular, trans-radial, or other approach, and once the catheter is positioned, such as advancing the catheter a previously deployed guidewire, advancing the implant removal device 380 along the catheter to the previously deployed fixation device. This simplifies the implant management tool and provides an implant removal device that both cuts and cauterizes the leaflets during fixation device removal.

As illustrated, the capture structure 380 can be utilize a biopolar or monopolar technique to delivery RF energy to the tissue. The capture structure 380 can include a conductive portion 400 and an insulative portion 402 that is closer to the elongate body 380. For instance, one or more distal coils of the coil 386 form the conductive portion 400 acting as an RF electrode, while the remainder of the coils 386 are insulated. A proximal end of the implant removal device 324 is electrically connected to an RF generator 404 that provides the RF energy to the conductive portion 400. Once the capture structure 382 is positioned around at least a portion of the fixation device, and tissue, and optionally tensioned as discussed above, activation of the RF generator 404 delivers RF energy to the tissue, thereby separating or cutting and allowing withdrawing of the released fixation device and capturing within the catheter.

Turning to FIG. 23, illustrated is another alternate embodiment of the implant removal device 480 according to the present invention. For simplicity, like reference numerals are associated with like structures and the discussion of other implant removal devices herein is also applicable to this configuration.

As mentioned previously, the implant removal device can include optional cutting surfaces 500. While discussion was provided of the tip 88 that can penetrate tissue in the configuration of FIGS. 17-21, one or more cutting surfaces 500 are formed on the coils 486. The cutting surfaces 500 can be sharpened edges 502, such as at the corners or surfaces of coils having the cross-section illustrated in FIGS. 10A-10F. Alternatively, instead of including a continuous cutting surface 500, and associated sharpened edges 502, the cutting surfaces can be discrete or non-continuous, such as one more projections, barbs, etc. that can be can be uniformly or non-uniformly distributed or arranged in a particular manner, and optionally having different sizes and shapes. With the optional cutting surfaces, such as cutting surface 500, torqueing the elongate body rotates the capture structure and cuts or separates tissue surrounding the fixation device 10.

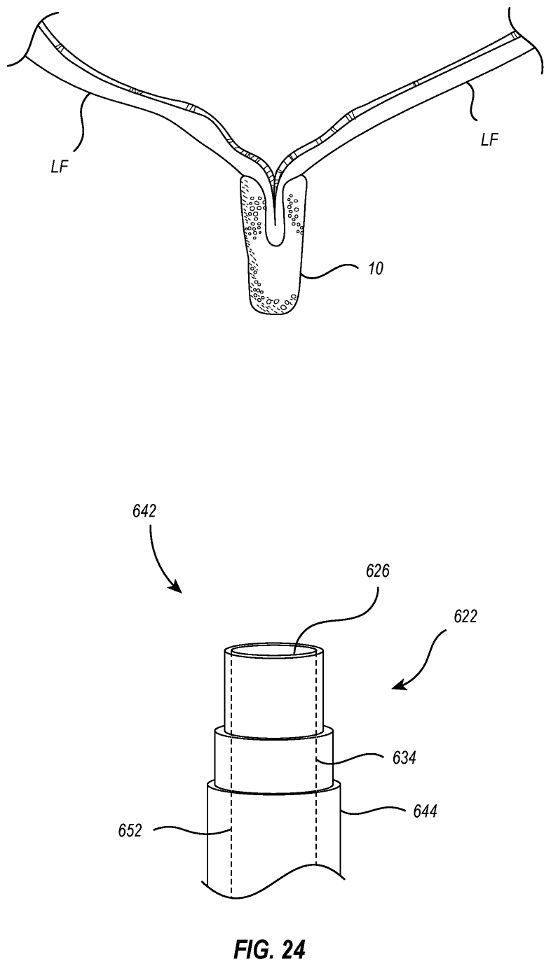

Turning to FIG. 24 illustrated is another configuration of an implant management tool. For simplicity, like reference numerals are associated with like structures and the discussion of other implant management tool herein is also applicable to this configuration.

As referenced previously in the configuration of FIG. 6A-B, with the opening 26 being elongate along a longitudinal axis 48 of the shaft 44, a greater space can be provided to capture the fixation device 10 (FIGS. 3-5) and tissue than if the opening were, for example, coaxial with the lumen of the shaft. FIG. 24 illustrates implant management tool 622 with an opening 626 formed in a distal end 642, with a cutting member 634 coaxial with the lumen 652. The operation and use of the implant management tool 622 is similar to that of the implant management tool 22 except that the fixation device is drawn into the lumen 652 through the opening 626 at a distal-most end of a shaft 644 instead of through the side.

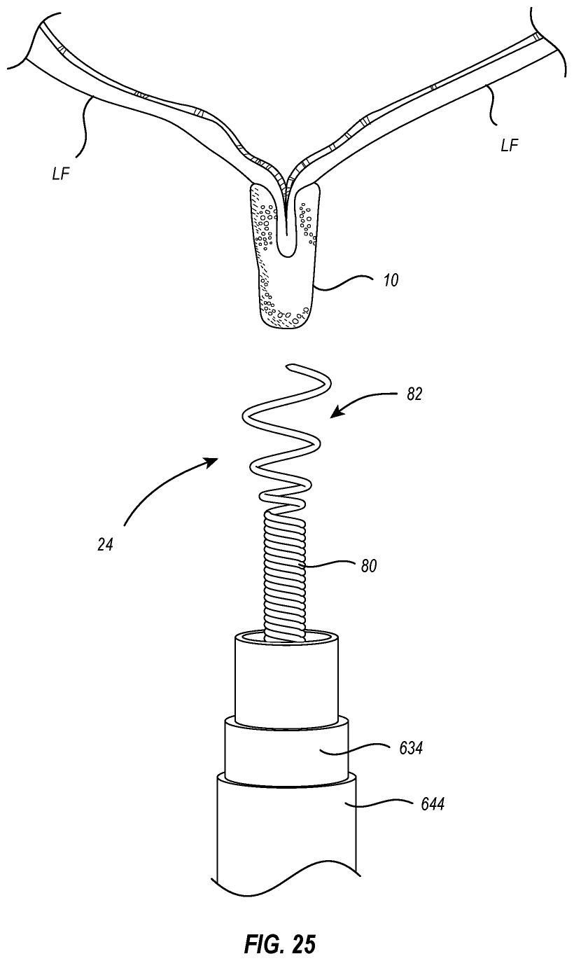

As illustrated in FIGS. 25-28 is a series of drawings illustrating the deployment of the implant management tool and the implant removal device and subsequent capture and removal of a previously deployed fixation device, such as the fixation device 10 (FIGS. 3-5). For simplicity, FIGS. 25-28 illustrate a portion of the anatomy from FIGS. 14-16 and a portion of each of the implant management tool and the implant removal device.

As illustrated in FIG. 25, the implant management tool 622 is advanced toward the previously deployed fixation device 10. With the opening 626 being generally orientated toward the deployed fixation device 10, the implant removal tool 24 can be translated or moved along the lumen 652 so the capture structure 82 is advanced toward the deployed fixation device 10, as illustrated in FIG. 25. This can be achieved, with reference to FIG. 6A-B, by manipulating the proximal end 40 of the implant removal tool 24 either directly or by way of the torque handle 74, as described in relation to FIG. 6A-B.

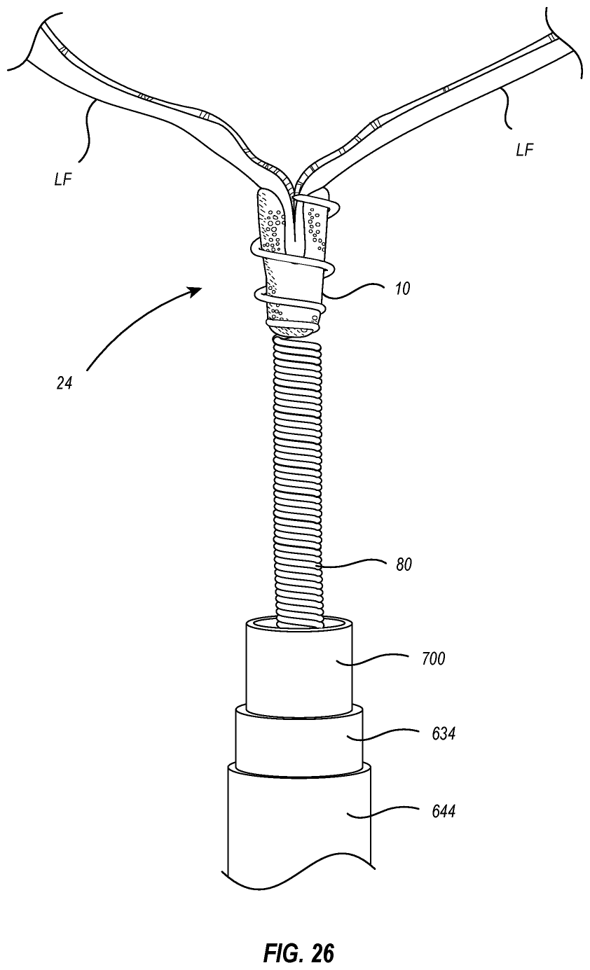

A user can further manipulate the proximal end 40 (FIG. 6A-B) to advance the capture structure 82 over the fixation device 10, as illustrated in FIG. 26. As the user advances the capture structure 82 towards the fixation device 10, such as by torqueing the elongate body 80 to rotate the capture structure 82 and advance the capture structure 82 along a portion of tissue surrounding the fixation device 10, the conical shape of the capture structure 82 begins to axially align the fixation device 10 with a longitudinal axis of the elongate body 80. For instance, as the implant removal tool 24 is rotated, the coils 86 engage with the tissue, applying a compressive force against the tissue and the fixation device 10 and draw the fixation device 10 and tissue into the capture opening 26. Optionally, the tip 88 can penetrate the tissue so the coils 86 penetrate the tissue, to provide enhanced capturing of the previously deployed fixation device 10 through both compressive force and mechanical engagement with the tissue.

When the capture structure 82 has been advanced sufficiently along the length of the fixation device 10, such that the last coil of the capture structure 82 is positioned at close to or past an end of the fixation device 10 closest to the leaflets, a physician or clinician can verify its location by fluoroscopy, intracardiac echocardiogram (ICE), or three-dimensional (3D) electroanatomical mapping (EAM) systems. Because the capture structure 82 is formed of a radiopaque material, the physician or clinician can verify the location with relative ease.

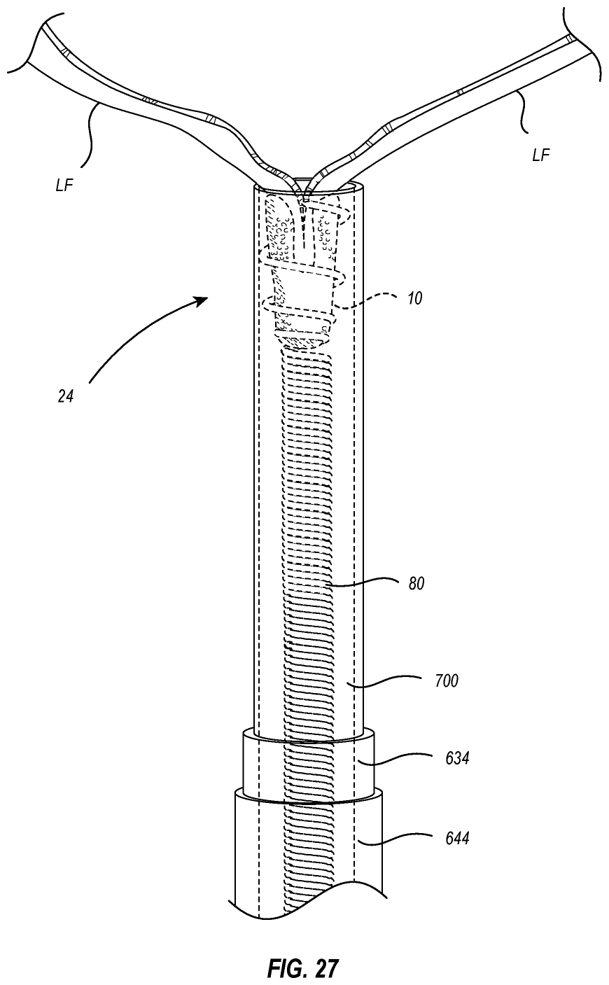

Once verified, the implant removal device 24 is drawn proximally to apply tension to the capture structure 82, as illustrated in FIG. 27. This tension can at least partially increase a length of the capture structure 82, including increasing a pitch of the coils of the capture structure 82, and increase a compressive force on the fixation device 10 and the tissue. The compressive force can be a transverse force that is applied transversely to a longitudinal axis of the capture structure 82.

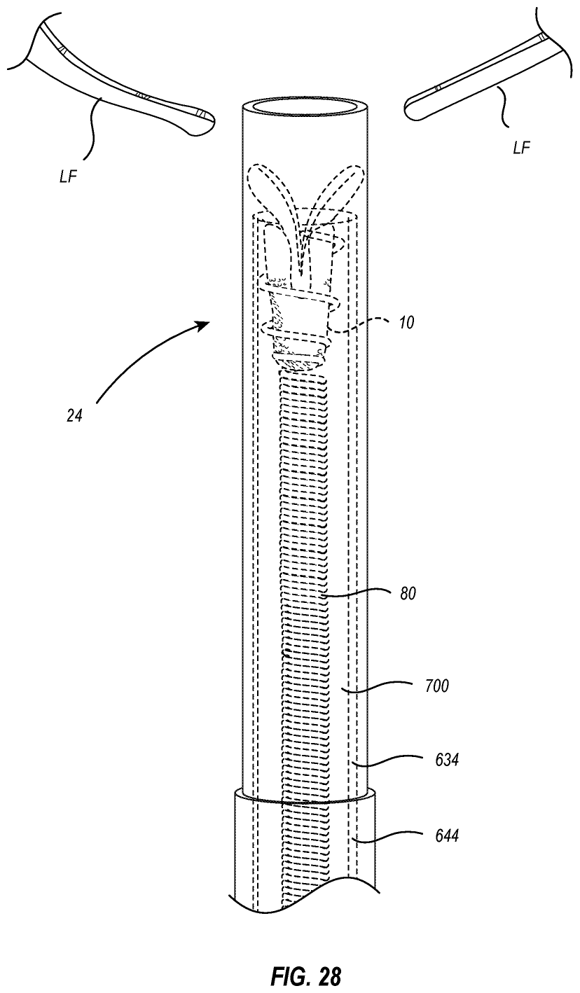

With the tissue partially taught, the sheath 700, such as sheath 100 from FIG. 9, can be advanced over the capture fixation device 10, as illustrated in FIG. 27. With the fixation device 10 restrained with the sheath 700 manipulation of the actuator 32 of the handle 30 can translate, and optionally rotate, the cutting member 634 to at least cut or separate the fixation device 10 from the tissue surrounding the fixation device 10, as illustrated in FIG. 28. For instance, the actuator 32 advances the cutting member 634 with an annular cutting edge to cut the tissue. The detached fixation device 10 is retained within the lumen 652. Once the fixation device 10 is detached from the surrounding tissue, the implant removal system, with the captured fixation device 10 can be removed from the patient.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

Following are some further example embodiments of the invention. These are presented only by way of example and are not intended to limit the scope of the invention in any way. Further, any example embodiment can be combined with one or more of the example embodiments.

Embodiment 1. An implant removal device including an elongate body having a proximal end and a distal end, the elongate body being resiliently flexible and configured to transmit torque from the proximal end to the distal end with a predetermined turning ratio; and a capture structure extending distally from the distal end and having a capture region, the capture structure being configured to selectively center a deployed implant in relation to a longitudinal axis of the elongate body and the capture region to aid with capture and subsequent removal.

Embodiment 2. The implant removal device of embodiment 1, further including an outer sheath disposed about at least a portion of the elongate body and being selectively translatable along a length of the elongate body and the capture structure.

Embodiment 3. The implant removal device of any of embodiments 1-2, wherein the elongate body comprises a stacked coil.

Embodiment 4. The implant removal device of any of embodiments 1-3, wherein the elongate body and the capture structure are formed of a coiled wired, the elongate body comprising a stacked coil portion and the capture structure comprising an open coil portion having a pitch greater than a pitch of the stacked coil portion.

Embodiment 5. The implant removal device of any of embodiments 1-4, wherein the elongate body comprises a hypotube with a plurality cuts that control a flexibility of the elongate body.

Embodiment 6. The implant removal device of any of embodiments 1-5, wherein the elongate body comprises a braided tubular member.

Embodiment 7. The implant removal device of any of embodiments 1-6, wherein the elongate body further comprises a polymeric jacket formed on an outer surface of the elongate body.

Embodiment 8. The implant removal device of any of embodiments 1-7, wherein the capture structure comprises at least one substantially resilient coil.

Embodiment 9. The implant removal device of any of embodiments 1-8, wherein the at least one substantially resilient coil comprises a sharpened tip.

Embodiment 10. The implant removal device of any of embodiments 1-9, wherein the at least one of substantially resilient coil comprises a cutting edge extending along at least a portion thereof.

Embodiment 11. The implant removal device of any of embodiments 1-10, wherein a material of the capture structure comprises a metal having a cross-section selected from circular, semi-circular, elliptical, oval, polygonal, ring, crescent, trefoil, and combinations or modifications thereof.

Embodiment 12. The implant removal device of any of embodiments 1-11, wherein the capture structure comprises an insulated portion and an exposed portion, the exposed portion being configured to transmit radiofrequency (RF) energy to tissue selectively contacting the exposed portion.

Embodiment 13. The implant removal device of any of embodiments 1-12, wherein the predetermined ratio is 1:1.

Embodiment 14. The implant removal device of any of embodiments 1-3, wherein the capture structure is configured to elongate under tension, the capture structure elongating in length by about 5% to about 30% of an original length of the capture structure.

Embodiment 15. The implant removal device of any of embodiments 1-14, wherein the capture structure is configured to apply a transverse force of about 0.9 Newtons to about 6.6 Newtons to the implant captured by the conical capture structure.

Embodiment 16. The implant removal device of any of embodiments 1-15, wherein the capture structure is configured to turn in relation to the elongate body.

Embodiment 17. The implant removal device of any of embodiments 1-16, wherein a junction of the capture structure and the elongate body is configured to facilitate movement of the capture structure in relation to the elongate body.

Embodiment 18. The implant removal device of any of embodiments 1-17, wherein the junction is cold-worked.

Embodiment 19. The implant removal device of any of embodiments 1-18, wherein material disposed at the junction has a cross-section smaller than at least one of the elongate body and the capture structure.

Embodiment 20. The implant removal device of any of embodiments 1-19, wherein, the capture structure has a capture structure distal end larger in cross-section than a capture structure proximal end.

Embodiment 21. The implant removal device of any of embodiments 1-20, wherein, the capture structure has a generally conical shape.

Embodiment 22. An implant removal system including an implant management tool configured for use in selectively separating an implant from tissue to which the implant is attached, the implant management tool comprising an opening configured to receive the implant and a cutting member configurated to cut the tissue; and an implant removal device selectively extending from the opening, the implant removable device including an elongate body having a proximal end and a distal end, the elongate body being resiliently flexible and configured to transmit torque from the proximal end to the distal end with a predetermined turning ratio; and a capture structure extending distally from the distal end and having a capture region, the capture structure being configured to selectively center a deployed implant in relation to a longitudinal axis of the elongate body and the capture region to aid with capture and subsequent removal.

Embodiment 23. The implant removal system of the embodiment 22, wherein the implant management tool includes a handle and a shaft extending distally from the handle.

Embodiment 24. The implant removal system of any of embodiments 22-23, wherein the opening is formed in a distal end region of a shaft extending from a handle.

Embodiment 25. The implant removal device of any of embodiments 22-24, wherein the cutting member is disposed within a shaft and is translatable to at least partially closes the opening.

Embodiment 26. The implant removal system of any of embodiments 22-25, wherein the implant removal device is slidably disposed within a lumen of the cutting member.

Embodiment 27. The implant removal system of any of embodiments 22-26, wherein the opening opens to a side of the shaft in a direction transverse to a longitudinal axis of the shaft.

Embodiment 28. The implant removal system of any of embodiments 22-27, wherein the open opens from an end of the shaft in a direction parallel to a longitudinal axis of the shaft.

Embodiment 29. The implant removal system of any of embodiments 22-28, wherein the implant removal device comprises an implant removable device from any one of claims 1-21.

Embodiment 30. A method of removing a fixation device including advancing an implant removal device towards an implant deployed on tissue, capturing the implant with the capture structure; and removing the implant from the patient. The implant removable device includes an elongate body having a proximal end and a distal end; and a capture structure extending distally from the distal end and having a capture region that selectively centers the implant in relation to a longitudinal axis of the elongate body and the capture region to aid with capture and subsequent removal.

Embodiment 31. The method of embodiment 30, further including positioning an implant management tool toward the tissue.

Embodiment 32. The method of any of embodiment 30-31, wherein capturing the implant further comprises torqueing the elongate body to rotate the capture structure and advance the capture structure along a portion of tissue surrounding the implant.

Embodiment 33. The method of any of embodiment 30-32, wherein the portion of the tissue surrounding the tissue is ingrowth tissue encapsulating the implant.

Embodiment 34. The method of any of embodiment 30-33, wherein capturing the implant further comprises torqueing the elongate body to rotate the capture structure and cut tissue surrounding the implant.

Embodiment 35. The method of any of embodiment 30-34, wherein torqueing the elongate body further comprises distally advancing the capture structure.

Embodiment 36. The method of any of embodiment 30-35, wherein capturing the implant further comprises applying a transverse force to the implant.

Embodiment 37. The method of any of embodiment 30-36, further including separating the implant from the tissue.

Embodiment 38. The method of any of embodiment 30-37, wherein the tissue is a valve leaflet.

Embodiment 39. The method of any of embodiment 30-38, wherein separating the implant from the tissue comprises applying radio frequency (RF) energy to the tissue.

Embodiment 40. The method of any of embodiment 30-39, wherein separating the implant from the tissue comprises distally advancing a cutting member with an annular cutting edge to cut the tissue.

Embodiment 41. The method of any of embodiment 30-40, applying tension to the capture structure to increase a transverse force applied to the tissue.

Embodiment 42. The method of any of embodiment 30-41, further including elongating the capture structure.

Embodiment 43. The method of any of embodiment 30-42, further including increasing a pitch between coils of the capture structure.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An implant removal device comprising:
an elongate body having a proximal end and a distal end, the elongate body being resiliently flexible and configured to transmit torque from the proximal end to the distal end with a predetermined turning ratio; and
a capture structure extending distally from the distal end and having a capture region, the capture structure being configured to selectively center a deployed implant in relation to a longitudinal axis of the elongate body and the capture region to aid with capture and subsequent removal.

2. The implant removal device of claim 1, further comprising an outer sheath disposed about at least a portion of the elongate body and being selectively translatable along a length of the elongate body and the capture structure.

3. The implant removal device of claim 1, wherein the elongate body comprises a stacked coil.

4. The implant removal device of claim 1, wherein the elongate body and the capture structure are formed of a coiled wired, the elongate body comprising a stacked coil portion and the capture structure comprising an open coil portion having a pitch greater than a pitch of the stacked coil portion.

5. The implant removal device of claim 1, wherein the elongate body comprises a hypotube with a plurality cuts that control a flexibility of the elongate body.

6. The implant removal device of claim 1, wherein the elongate body comprises a braided tubular member.

7. The implant removal device of claim 1, wherein the elongate body further comprises a polymeric jacket formed on an outer surface of the elongate body.

8. The implant removal device of claim 1, wherein the capture structure comprises at least one substantially resilient coil.

9. The implant removal device of claim 8, wherein the at least one substantially resilient coil comprises a sharpened tip.

10. The implant removal device of claim 8, wherein the at least one of substantially resilient coil comprises a cutting edge extending along at least a portion thereof.

11. The implant removal device of claim 1, wherein a material of the capture structure comprises a metal having a cross-section selected from circular, semi-circular, elliptical, oval, polygonal, ring, crescent, trefoil, and combinations or modifications thereof.

12. The implant removal device of claim 1, wherein the capture structure comprises an insulated portion and an exposed portion, the exposed portion being configured to transmit radiofrequency (RF) energy to tissue selectively contacting the exposed portion.

13. The implant removal device of claim 1, the predetermined ratio is 1:1.

14. The implant removal device of claim 1, wherein the capture structure is configured to elongate under tension, the capture structure elongating in length by about 5% to about 30% of an original length of the capture structure.

15. The implant removal device of claim 1, wherein the capture structure is configured to apply a transverse force of about 0.9 Newtons to about 6.6 Newtons to the implant captured by the conical capture structure.

16. The implant of claim 1, wherein the capture structure is configured to turn in relation to the elongate body.

17. The implant removal device of claim 1, wherein a junction of the capture structure and the elongate body is configured to facilitate movement of the capture structure in relation to the elongate body.

18. The implant removal device of claim 17, wherein the junction is cold-worked.

19. The implant removal device of claim 17, wherein material disposed at the junction has a cross-section smaller than at least one of the elongate body and the capture structure.

20. The implant removal device of claim 1, wherein, the capture structure has a capture structure distal end larger in cross-section than a capture structure proximal end.

21. The implant removal device of claim 1, wherein, the capture structure has a generally conical shape.

22. An implant removal system comprising:
an implant management tool configured for use in selectively separating an implant from tissue to which the implant is attached, the implant management tool comprising an opening configured to receive the implant and a cutting member configurated to cut the tissue; and
an implant removal device selectively extending from the opening, the implant removable device comprising:
an elongate body having a proximal end and a distal end, the elongate body being resiliently flexible and configured to transmit torque from the proximal end to the distal end with a predetermined turning ratio; and
a capture structure extending distally from the distal end and having a capture region, the capture structure being configured to selectively center a deployed implant in relation to a longitudinal axis of the elongate body and the capture region to aid with capture and subsequent removal.

23. The implant removal system of claim 22, wherein the implant management tool comprises a handle and a shaft extending distally from the handle.

24. The implant removal system of claim 22, wherein the opening is formed in a distal end region of a shaft extending from a handle.

25. The implant removal system of claim 22, wherein the cutting member is disposed within a shaft and is translatable to at least partially closes the opening.

26. The implant removable system of claim 25, wherein the implant removal device is slidably disposed within a lumen of the cutting member.

27. The implant removal system of claim 25, wherein the opening opens to a side of the shaft in a direction transverse to a longitudinal axis of the shaft.

28. The implant removal system of claim 25, wherein the open opens from an end of the shaft in a direction parallel to a longitudinal axis of the shaft.

29. The implant removal system of claim 22, wherein the implant removal device comprises an implant removable device from any one of claims 1-21.

30. A method of removing an implant, the method comprising:
advancing an implant removal device towards an implant deployed on tissue, the implant removable device comprising:
an elongate body having a proximal end and a distal end; and
a capture structure extending distally from the distal end and having a capture region that selectively centers the implant in relation to a longitudinal axis of the elongate body and the capture region to aid with capture and subsequent removal;
capturing the implant with the capture structure; and
removing the implant from the patient.

31. The method of claim 30, further comprising positioning an implant management tool toward the tissue.

32. The method of claim 30, wherein capturing the implant further comprises torqueing the elongate body to rotate the capture structure and advance the capture structure along a portion of tissue surrounding the implant.

33. The method of claim 32, wherein the portion of the tissue surrounding the tissue is ingrowth tissue encapsulating the implant.

34. The method of claim 30, wherein capturing the implant further comprises torqueing the elongate body to rotate the capture structure and cut tissue surrounding the implant.

35. The method of claim 34, wherein torqueing the elongate body further comprises distally advancing the capture structure.

36. The method of claim 30, wherein capturing the implant further comprises applying a transverse force to the implant.

37. The method of claim 30, further comprises separating the implant from the tissue.

38. The method of claim 37, wherein the tissue is a valve leaflet.

39. The method of claim 37, wherein separating the implant from the tissue comprises applying radio frequency (RF) energy to the tissue.

40. The method of claim 37, wherein separating the implant from the tissue comprises distally advancing a cutting member with an annular cutting edge to cut the tissue.

41. The method of claim 30, further comprises applying tension to the capture structure to increase a transverse force applied to the tissue.

42. The method of claim 41, further comprising elongating the capture structure.

43. The method of claim 42, further comprising increasing a pitch between coils of the capture structure.
